(19) United States
(12) Patent Application Publication
(10) Pub. No.: US 2023/0036728 A1
(43) Pub. Date: Feb. 2, 2023

(54) SYSTEMS AND METHODS OF REDUCING IN-HOSPITAL MORTALITY RATE

(71) Applicant: Neuravi Limited, Galway (IE)

(72) Inventors: Rahul KHANNA, Naperville, IL (US); Shelly IKEME, West Chester, PA (US); Emilie KOTTENMEIER, Laguna Beach, CA (US); Alia KHALED, Burlington (CA); Eamon BRADY, Loughrea (IE); David VALE, Barna (IE); Michael GILVARRY, Headford (IE); Mahmood K. RAZAVI, Irvine, CA (US); John O'Shaughnessy, Galway (IE); Brendan CASEY, Galway (IE); David HARDIMAN, Dublin (IE); Kevin McARDLE, Furlough (IE); Patrick GRIFFIN, Castlegar (IE); Jason McNAMARA, Knock (IE)

(73) Assignee: Neuravi Limited, Galway (IE)

(21) Appl. No.: 17/964,605

(22) Filed: Oct. 12, 2022

Related U.S. Application Data
(63) Continuation of application No. 17/870,995, filed on Jul. 22, 2022.
(60) Provisional application No. 63/225,245, filed on Jul. 23, 2021.

(51) Int. Cl. A61B 17/221; G16H 20/40

(52) U.S. Cl. CPC A61B 17/221; G16H 20/40

(57) ABSTRACT

Method for using a clot retrieval device for treating a clot in a blood vessel for use in the treatment of ischemic stroke to reperfuse an obstructed vessel. Use of the clot retrieval device reducing in-hospital mortality.

### CROSS-REFERENCE TO RELATED APPLICATION

This application is a continuation application of U.S. patent application Ser. No. 17/870,995, filed 22 Jul. 2022, which claims the benefit of U.S. Provisional Application No. 63/225,245, filed on 23 Jul. 2021, which is incorporated herein by reference in its entirety as if fully set forth below.

### FIELD

This disclosure relates to devices and methods of removing acute blockages from blood vessels.

### BACKGROUND

The World Health Organization estimates that 15,000,000 blood clots occur annually. Clots may develop and block vessels locally without being released in the form of an embolus—this mechanism is common in the formation of coronary blockages. Acute obstructions may include blood clots, misplaced devices, migrated devices, large emboli and the like. Thromboembolism occurs when part or all of a thrombus breaks away from the blood vessel wall. This clot is then carried in the direction of blood flow. The large vessels of the brain include the Internal Carotid Artery (ICA), Middle Cerebral Artery (MCA), Vertebral Artery (VA), and the Basilar Artery (BA). Clots can include a range of morphologies and consistencies. Long strands of softer clot material may tend to lodge at bifurcations or trifurcations, resulting in multiple vessels being simultaneously occluded over significant lengths. Older clot material can also be less compressible than softer fresher clots, and under the action of blood pressure it may distend the compliant vessel in which it is lodged. Clots may also vary greatly in length, even in any one given area of the anatomy. For example, clots occluding the middle cerebral artery of an ischemic stroke patient may range from just a few millimeters to several centimeters in length.

Of the 15,000,000 clots that occur annually, one-third of patients die and another one-third are disabled. Two of the primary factors associated with mortality in these patients are the occlusion location and the time to treatment. Large-vessel occlusions present in 46% of unselected acute stroke patients presenting in academic medical centers, are associated with higher stroke severity. These more proximal vessels feed a large volume of brain tissue, ergo clinicians use the presenting NIHSS (National Institute of Health Stroke Scale) score as an indicator of large-vessel occlusion.

With this, it is understood that an ischemic stroke may result if the clot lodges in the cerebral vasculature. It is estimated that 87% of stroke cases are acute ischemic stroke (AIS). In the United States alone, roughly 700,000 AIS cases occur every year and this number is expected to increase with an ageing population. Occlusion of these large arteries in ischemic stroke is associated with significant disability and mortality. Revascularization of intracranial artery occlusions is the therapeutic goal in stroke therapy. Endovascular mechanical revascularization (thrombectomy) is an increasingly used method for intracranial large vessel recanalization in acute stroke. Currently, a number of mechanical recanalization devices are in clinical use. First generation devices included the Merci Retriever device. Newer devices based on stent-like technology, referred to as “stentrievers” or “stent-retrievers”, are currently displacing these first generation thrombectomy devices for recanalization in acute ischemic stroke.

Several randomized clinical trials have demonstrated that mechanical thrombectomy using stent-like clot retriever devices are a safe and effective treatment to remove clots from cerebral vessels of acute stroke patients, but such devices are not without disadvantages. A stent-like clot retriever relies on its outward radial force to grip the clot. If the radial force is too low, the device will lose its grip on the clot. If the radial force is too high, the device may damage the vessel wall and may require too much force to withdraw. Such devices that have sufficient radial force to deal with all clot types may therefore cause vessel trauma and serious patient injury, and retrievers that have appropriate radial force to remain atraumatic may not be able to effectively handle all clot types. In this respect, retriever devices may differ in size, shape, and physical properties, such as radial force, as discussed above, ease of deployment, friction, radiopacity and interaction with vessel wall. See, Loh Y, Jahan R, McArthur D. Recanalization rates decrease with increasing thrombectomy attempts. American Journal of Neuroradiology. 2010 May; 31(5):935-9; and Arai D, Ishii A, Chihara H, Ikeda H, Miyamoto S. Histological examination of vascular damage caused by stent retriever thrombectomy devices, J Neurointery Surg. 2016 October; 8(10):992-5. Some designs have also been based on in-vitro stroke models that incorporate realistic clot analogs derived from animal blood that represent the wide range of human clots retrieved from stroke patients. See, Eugene F, Gauvrit J-Y, Ferré J-C, Gentric J-C, Besseghir A, Ronzière T, et al. One-year MR angiographic and clinical follow-up after intracranial mechanical thrombectomy using a stent retriever device, AJNR Am J Neuroradiol. 2015 January; 36(1):126-32 (18), each of which are incorporated by reference herein in their entirety.

Currently, intravenous (IV) lytics are used for patients presenting up to 4.5 hours after symptom onset. Current guidelines recommend administering IV lytics in the 3-4.5 hour window to those patients who meet the ECASS 3 (European Cooperative Acute Stroke Study 3) trial inclusion/exclusion criteria. Since a large percentage of strokes presenting at hospitals are large vessel occlusions, this is an important clinical challenge to address. Additionally, not all patients may be treated with thrombolytic therapy, and so mechanical thrombectomy is a valuable alternative in patients contraindicated to t-PA (tissue plasminogen activator) or where t-PA treatment was not effective.

Further, acute stroke treatment protocols vary by hospital center. Often, CT is used to exclude hemorrhagic stroke, and CT Angiography is used. Additional imaging assessment, such as MRI or CT Perfusion, varies by center. Recent AIS trials have demonstrated the clinical benefit and reperfusion efficacy of endovascular therapy using stent-retriever devices. See Zaidat O O, Castonguay A C, Gupta R, Sun C J, Martin C, Holloway W E, et al. The first pass effect: a new measure for stroke thrombectomy devices. Stroke. 2018; 49; 660-666; Chueh J Y, Marosfoi M G, Brooks O W, King R M, Puri A S, Gounis M J. Novel distal emboli protection technology: the EmboTrap. Intery Neurol. 2017; 6:268-276. doi: 10.1159/000480668; Kabbasch C, Mpotsaris A, Liebig T, Soderman M, Holtmannspotter M, Cronqvist M, et al. TREVO 2 Trialists. Trevo versus Merci retrievers for thrombectomy revascularisation of large vessel occlusions in acute ischaemic stroke (TREVO 2): a randomised trial. Lancet. 2012; 380:1231-1240. doi: 10.1016/S0140-6736(12)61299-9. There are several FDA approved stent retriever devices indicated for neuro-thrombectomy, including Merci®, Trevo®, and Solitaire®. These devices are generally described in U.S. Pat. Nos. 8,066,757; 8,088,140; 8,945,172; 9,320,532; 8,585,713; 8,945,143; 8,197,493; 8,940,003; 9,161,766; 8,679,142; 8,070,791; 8,574,262; 9,387,098; 9,072,537; 9,044,263; 8,795,317; 8,795,345; 8,529,596; and 8,357,179. Presently, these devices are now considered the standard of care for treatment of AIS secondary to large-vessel occlusion. See, Powers W J, Derdeyn C P, Biller J, Coffey C S, Hoh B L, Jauch E C, et al; American Heart Association Stroke Council. 2015 American Heart Association/American Stroke Association focused update of the 2013 guidelines for the early management of patients with acute ischemic stroke regarding endovascular treatment: a guideline for healthcare professionals from the American Heart Association/American Stroke Association. Stroke. 2015; 46:3020-3035. doi: 10.1161/STR.0000000000000074.

Two multi-center studies using a specific FDA approved thrombectomy device generally described in U.S. Pat. Nos. U.S. Pat. Nos. 8,777,976; 8,852,205; 9,402,707; 9,445,829; and 9,642,639, demonstrated high reperfusion rates (modified treatment in cerebral ischemia (mTICI)≥2b within three passes without rescue) of 75.0% and 80.2%, respectively. See Zaidat O O, Bozorgchami H, Ribo M, et al. Primary Results of the Multicenter ARISE II Study (Analysis of Revascularization in Ischemic Stroke With EmboTrap). Stroke 2018; 49:1107-15; Mattle H P, Scarrott C, Claffey M, et al. Analysis of revascularisation in ischaemic stroke with EmboTrap (ARISE I study) and meta-analysis of thrombectomy. Intery Neuroradiol 2019; 25:261-70. These trials had prespecified patient inclusion and exclusion criteria, and thus patient outcomes may differ from real-world use of the stent retriever device used in those studies. Two U.S. studies assessing hospital outcomes among patients who underwent endovascular treatment for acute ischemic stroke using two other thrombectomy devices using real-world data reported outcomes parameters such as hospital length of stay (LOS) and hospital costs. See Rai A T, Crivera C, Kalsekar I, et al. Endovascular Stroke Therapy Trends From 2011 to 2017 Show Significant Improvement in Clinical and Economic Outcomes. Stroke 2019a; 50:1902-06; Rai A T, Crivera C, Kottenmeier E, et al. Outcomes associated with endovascular treatment among patients with acute ischemic stroke in the USA. J Neurointery Surg 2019b; 12:422-26.

No studies have evaluated real-world hospital resource utilization, economic costs, and mortality associated with the use of the specific FDA approved thrombectomy device in patients with AIS. The solution of this disclosure resolves these and other issues of the art.

### SUMMARY

The subject of this disclosure is the use of a clot retrieval device to treat ischemic stroke for restoring perfusion and/or removing a clot and other obstructions from the neurovascular arteries and veins as well as other vascular beds.

An example method or use of treating an ischemic stroke can include delivering a first revascularization device to a blood vessel of a respective human patient of a first plurality of human patients for retrieving a thrombus, restoring perfusion to the blood vessel by passing the first revascularization device by, through, or about the thrombus, and removing the first revascularization device. The method or use can achieve, by the first revascularization device, approximately a 9.7% in-hospital mortality rate for the first plurality of human patients with one or more cerebral occlusions within a predetermined time period.

An example method or use can further include delivering a second revascularization device to a blood vessel of a respective human patient of a second plurality of human patients for retrieving a thrombus, restoring perfusion to the blood vessel by passing the second revascularization device by, through, or about the thrombus, and removing the second revascularization device. The method or use can reduce in-hospital mortality, by the first revascularization device, by approximately 11.9% for the first plurality of human patients with one or more cerebral occlusions within a predetermined time period.

An example method or use can include reducing in-hospital mortality, by the first revascularization device, by approximately 3.1% for the first plurality of human patients with one or more cerebral occlusions within a predetermined time period.

An example first revascularization device of the method or use can include a framework of struts forming a porous inner body flow channel and having a tubular main body portion and a distal end. The example first revascularization device can also include a framework of struts forming an outer tubular body radially surrounding the tubular main body portion of the inner body during both the collapsed delivery configuration and the expanded deployed configuration.

An example method or use can include delivering a first revascularization device to a blood vessel a respective human patient of a plurality of human patients for retrieving a thrombus, restoring perfusion to the blood vessel by passing the first revascularization device by, through, or about the thrombus, and removing the first revascularization device. The method or use can achieve, by the first revascularization device, approximately 16.1% 30-day all-cause readmission rate for the plurality of human patients with one or more cerebral occlusions within a predetermined time period.

In some examples, the method or use can achieve, by the first revascularization device, approximately 10.8% 30-day cardiovascular related readmission rate for the plurality of human patients with one or more cerebral occlusions within a predetermined time period.

In some examples, the method or use can achieve, by the first revascularization device, approximately 4.3% 30-day acute ischemic stroke related readmission rate for the plurality of human patients with one or more cerebral occlusions within a predetermined time period.

In some examples, the method or use can achieve, by the first revascularization device, approximately $45,782 USD total cost of hospitalization care for the first plurality of human patients with one or more cerebral occlusions within a predetermined time period.

In some examples, the method or use can further include delivering a second revascularization device to a blood vessel of a respective human patient of a second plurality of human patients for retrieving a thrombus, restoring perfusion to the blood vessel by passing the second revascularization device by, through, or about the thrombus, and removing the second revascularization device. The method or use can reduce in-hospital mortality, by the first revascularization device, from approximately 11.9% to approximately 3.1% for the first plurality of human patients with one or more cerebral occlusions within a predetermined time period.

In some examples, the method or use can achieve, by the first revascularization device, approximately a 9.7% in-hospital mortality rate for the first plurality of human patients with one or more cerebral occlusions within a predetermined time period.

In some examples, the method or use can further include achieving, by the first revascularization device, approximately 16.1% 30-day all-cause readmission rate for the first plurality of human patients with one or more cerebral occlusions within a predetermined time period.

In some examples, the method or use can further include achieving, by the first revascularization device, approximately 10.8% 30-day cardiovascular related readmission rate for the first plurality of human patients with one or more cerebral occlusions within a predetermined time period.

In some examples, the method or use can further include achieving, by the first revascularization device, approximately 4.3% 30-day acute ischemic stroke related readmission rate for the first plurality of human patients with one or more cerebral occlusions within a predetermined time period.

The example first revascularization device can include a framework of struts forming a porous inner body flow channel and having a tubular main body portion and a distal end, and a framework of struts forming an outer tubular body radially surrounding the tubular main body portion of the inner body during both the collapsed delivery configuration and the expanded deployed configuration.

An example method or use of treating an ischemic stroke can include delivering a device to a blood vessel of a respective human patient of a first plurality of human patients for retrieving a thrombus. The device can include a framework of struts forming a porous inner body flow channel and having a tubular main body portion and a distal end, and a framework of struts forming an outer tubular body radially surrounding the tubular main body portion of the inner body during both the collapsed delivery configuration and the expanded deployed configuration. The method or use can include restoring perfusion to the blood vessel by passing the device by, through, or about the thrombus, and removing the device. The method or use can achieve, by the device, an in-hospital survival rate of approximately 89.3% for the first plurality of human patients with one or more cerebral occlusions within a predetermined time period.

An example plurality of human patients can include inclusion criteria defined as patients who underwent a mechanical thrombectomy procedure for acute ischemic stroke between Jul. 1, 2018-Dec. 31, 2019 were identified from the Premier Healthcare Database.

An example plurality of human patients can include inclusion criteria defined as patients ≥18 years old at the time of index hospital admission.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 shows a patient catheterized via femoral access with an example clot retrieval device positioned in a cerebral vessel using the arterial system for its delivery.

FIG. 2 shows certain anatomy of cerebral arteries above the aortic arch leading to the brain.

FIG. 3 shows an isometric view of an example stent retriever device of this disclosure.

FIG. 4 is a flow diagram illustrating flow of enrollment in the first study of this disclosure.

FIG. 5 is a table summarizing patient demographics for the first study of this disclosure.

FIG. 6 a table summarizing hospital characteristics for the first study of this disclosure.

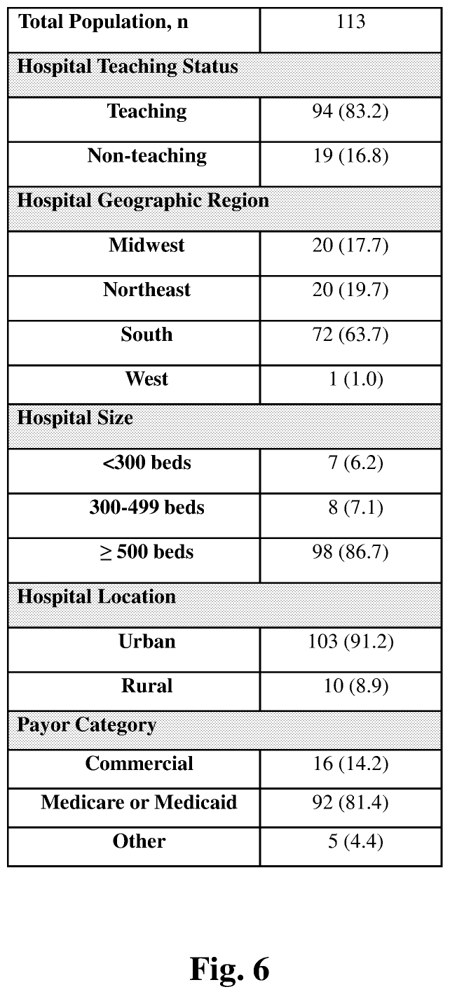

FIG. 7 is a table of outcomes for the first study of this disclosure.

FIG. 8 is a table of a comparison of results of the first study of this disclosure compared to other real-world studies in the U.S.

FIG. 9 is a flow diagram illustrating flow of enrollment in the second study of this disclosure.

FIG. 10 is a table summarizing patient demographics for the second study of this disclosure.

FIG. 11 a table summarizing hospital characteristics for the second study of this disclosure.

FIG. 12 is a table of outcomes for the second study of this disclosure.

FIG. 13 is a table of a comparison of results of the second study of this disclosure compared to other real-world studies in the U.S.

FIG. 14 is a flow diagram illustrating flow of enrollment in the third study of this disclosure.

FIG. 15 is a table summarizing patient demographics and hospital characteristics by mechanical thrombectomy volume that different significantly for the third study of this disclosure.

FIG. 16 is a table summarizing additional patient demographics and hospital characteristics by mechanical thrombectomy volume for the third study of this disclosure.

FIG. 17 is a table of adjusted outcomes by mechanical thrombectomy volume via generalized estimating equations (GEE) model regression analysis for the third study of this disclosure.

FIG. 18 is a table of study outcomes of the third study of this disclosure.

FIG. 19 depicts a graphical overview of a method of treating thrombus by mechanical thrombectomy according to this disclosure.

FIG. 20 depicts a graphical overview of a method of treating thrombus by mechanical thrombectomy according to this disclosure.

FIG. 21 depicts a graphical overview of a method of treating thrombus by mechanical thrombectomy according to this disclosure.

FIG. 22 depicts a graphical overview of a method of treating thrombus by mechanical thrombectomy according to this disclosure.

FIG. 23 depicts a graphical overview of a method of treating thrombus by mechanical thrombectomy according to this disclosure.

FIG. 24 depicts a graphical overview of a method of treating thrombus by mechanical thrombectomy according to this disclosure.

FIG. 25 depicts a graphical overview of a method of treating thrombus by mechanical thrombectomy according to this disclosure.

FIG. 26 depicts a graphical overview of a method of treating thrombus by mechanical thrombectomy according to this disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, vasculature of a “subject” or “patient” may be vasculature of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include a doctor, surgeon, or any other individual or delivery instrumentation associated with delivery of a clot retrieval device to the vasculature of a subject.

As discussed herein, “thrombus” can be understood as a clot in the circulatory system that remains in a site of the vasculature hindering or otherwise obstructing flow in a blood vessel. The terms, “clot”, “thrombus”, “obstruction”, “occlusion”, “blockage”, and/or the like, can be and are often used interchangeably throughout this disclosure.

Delivery of a “revascularization device” is typically accomplished via delivery of one or more catheters into the femoral artery and/or the radial artery, guided into the arteries of the brain, vascular bypass, angioplasty, and/or the like. “Revascularization devices” can include, but not be limited to, one or more stents, stentrievers, clot removal devices, clot retrieval devices, aspiration systems, one or more combinations thereof, and/or the like, each of which are often used interchangeably throughout this disclosure.

As discussed herein, “mTICI” means modified thrombolysis in cerebral infarction (TICI) score. An mTICI score of 0 means no perfusion. An mTICI score of 1 means antegrade reperfusion past the initial occlusion but limited distal branch filling with little or slow distal reperfusion. An mTICI score of 2 generally means incomplete antegrade reperfusion wherein the contrast passes the occlusion and opacifies the distal arterial bed but there are residual antegrade perfusion deficits. More particularly, an mTICI score of 2a means antegrade reperfusion of less than half of the occluded target artery previously ischemic territory (e.g., in 1 major division of the MCA and its territory). An mTICI score of 2b means antegrade reperfusion of more than half of the previously occluded target artery ischemic territory (e.g., in 2 major divisions of the MCA and their territories). An mTICI score of 2c means antegrade reperfusion of >90% but less than TICI 3 or near complete reperfusion. An mTICI score of 3 means full perfusion with filling of all distal branches.

It is noted, however, that other measures of cerebral scoring standards, such as expanded TICI (eTICI), other known and/or to-be-developed cerebral scoring standards, provide measures of cerebral scoring and are thus directly and/or indirectly applicable in understanding scope of the presently disclosed solution. eTICI scale is a 7-point compilation of TICI grades that reflects all previously reported thresholds used to define reperfusion after endovascular stroke therapy. For example, eTICI grade 0, just as mTICI, can be equivalent to no reperfusion or 0% filling of the downstream territory. eTICI 1 can indicate thrombus reduction without any reperfusion of distal arteries, including reperfusion of less than half or 1-49%. eTICI of 2b50 can be 50-66% reperfusion. eTICI 2b67 can be 67-89% reperfusion, exceeding TICI but below TICI2C. eTICI 2c can be equivalent to TICI 2C or 90-99% reperfusion. eTICI 3 can be complete or 100% reperfusion, such as TICI 3. It is understood that one of ordinary skill in the art can also correlate between currently known cerebral scoring standards and/or to-be-developed cerebral scoring standards (e.g., from mTICI to eTICI).

As discussed herein, “NIHSS Score” means The National Institutes of Health Stroke Scale, or NIH Stroke Scale (NIHSS) and is a tool used by healthcare providers to objectively quantify the impairment caused by a stroke. The NIHSS is composed of 11 items, each of which scores a specific ability between a 0 and 4. For each item, a score of 0 typically indicates normal function in that specific ability, while a higher score is indicative of some level of impairment. The individual scores from each item are summed in order to calculate a patient's total NIHSS score. The maximum possible score is 42, with the minimum score being a 0.

As discussed herein, “mRS” means the modified Rankin Scale (mRS) that is a commonly used scale for measuring the degree of disability or dependence in the daily activities of people who have suffered a stroke or other causes of neurological disability. The mRS scale runs from 0-6, running from perfect health without symptoms to death. An mRS score of 0 is understood as no symptoms being observed. An mRS score of 1 is understood as no significant disability is observed and the patient is able to carry out all usual activities, despite some symptoms. An mRS score of 2 is understood as slight disability and the patient is able to look after own affairs without assistance, but unable to carry out all previous activities. An mRS score of 3 is understood as moderate disability whereby the patient can require some help but is able to walk unassisted. An mRS score of 4 is understood as moderate severe disability and the patient is unable to attend to own bodily needs without assistance or walk unassisted. An mRS score of 5 is understood as severe disability and the patient requires constant nursing care and attention, bedridden, incontinent. An mRS score of 6 is understood as the patient being deceased.

As discussed herein, the term “safety”, as it relates to a clot retrieval device, delivery system, or method of treatment refers to a relatively low severity of adverse events, including adverse bleeding events, infusion or hypersensitivity reactions. Adverse bleeding events can be the primary safety endpoint and include, for example, major bleeding, minor bleeding, and the individual components of the composite endpoint of any bleeding event.

As discussed herein, unless otherwise noted, the term “clinically effective” (used independently or to modify the term “effective”) can mean that it has been proven by a clinical trial wherein the clinical trial has met the approval standards of U.S. Food and Drug Administration, EMEA or a corresponding national regulatory agency. For example, a clinical study may be an adequately sized, randomized, double-blinded controlled study used to clinically prove the effects of the reperfusion device and related systems of this disclosure. Most preferably to clinically prove the effects of the reperfusion device with respect to an ischemic event, for example, to achieve a clinically effective outcome in for the patient suffering the ischemic event (e.g., mRS less than or equal to 2) and/or achieve reperfusion the vessel(s) afflicted by the ischemic event.

As discussed herein, “sICH” is any extravascular blood in the brain or within the cranium associated with clinical deterioration, as defined by an increase of 4 points or more in the score on the NIHSS, or that leads to death and is identified as the predominant cause of the neurologic deterioration. For the purpose of this disclosure, subjects with sICH identified through all post-treatment scans up to the 24-hour time-point (including those performed due to clinical deterioration), were considered in the study discussed herein.

As discussed herein, the term “computed tomography” or CT means one or more scans that make use of computer-processed combinations of many X-ray measurements taken from different angles to produce cross-sectional (tomographic) images (virtual “slices”) of specific areas of a scanned object, allowing the user to see inside the object without cutting. Such CT scans of this disclosure can refer to X-ray CT as well as many other types of CT, such as positron emission tomography (PET) and single-photon emission computed tomography (SPECT).

As used herein, the term “odds ratio” or OR means the strength of an association between two events. In general, two events are independent if and only if the OR equals 1.0 (e.g., the ratio of odds of one event are the same in either the presence or absence of the other event).

The present disclosure is related to systems, methods and devices restoring perfusion in blood vessels, and in particular clots from cerebral vessels. Certain features, such as a capture net, can be designed to trap a wide range of clot compositions inside the device, and an inner channel to stabilize the clot during retrieval. Certain feature of the retriever of this disclosure can allow the segments to remain open and opposed to the vessel wall while retracted through challenging vessels.

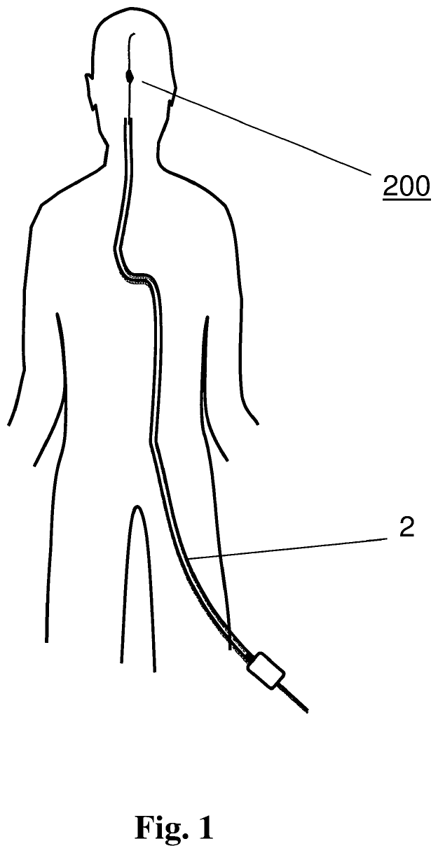

As an example, FIG. 1 depicts a schematic representation of the catheterization of a patient with a clot retrieval device 200, also known as a reperfusion device, via the femoral artery with a catheter 2. Example device 200 is a clinically approved FDA clot retrieval device that can restore blood flow in the neurovasculature by removing thrombus in patients experiencing ischemic stroke within 8 hours of symptom onset. However, it is understood that example device 200 could be used to restore blood flow in less than 8 hours of symptom onset (e.g., 6 hours) or up to 24 hours from symptom onset. As applicable procedure guidelines change with respect to the use of clot retrieval devices for treatment of ischemic events, it is also conceivable that device 200 could be used more than 24 hours from symptom onset. Device 200 can be understood as including features are clearly described in U.S. Pat. Nos. 8,777,976; 8,852,205; 9,402,707; 9,445,829; and 9,642,639, each of which are incorporated by reference in their entirety into this application as if set forth in full and attached in the appendix to priority application U.S. 63/225,245. Note that reperfusion devices can also be introduced through the wrist artery (radial access) or directly through the carotid artery. While both radial and carotid access avoids the aortic arches, there are other drawbacks. However, all three approaches are considered to be known to ones of skill in this art.

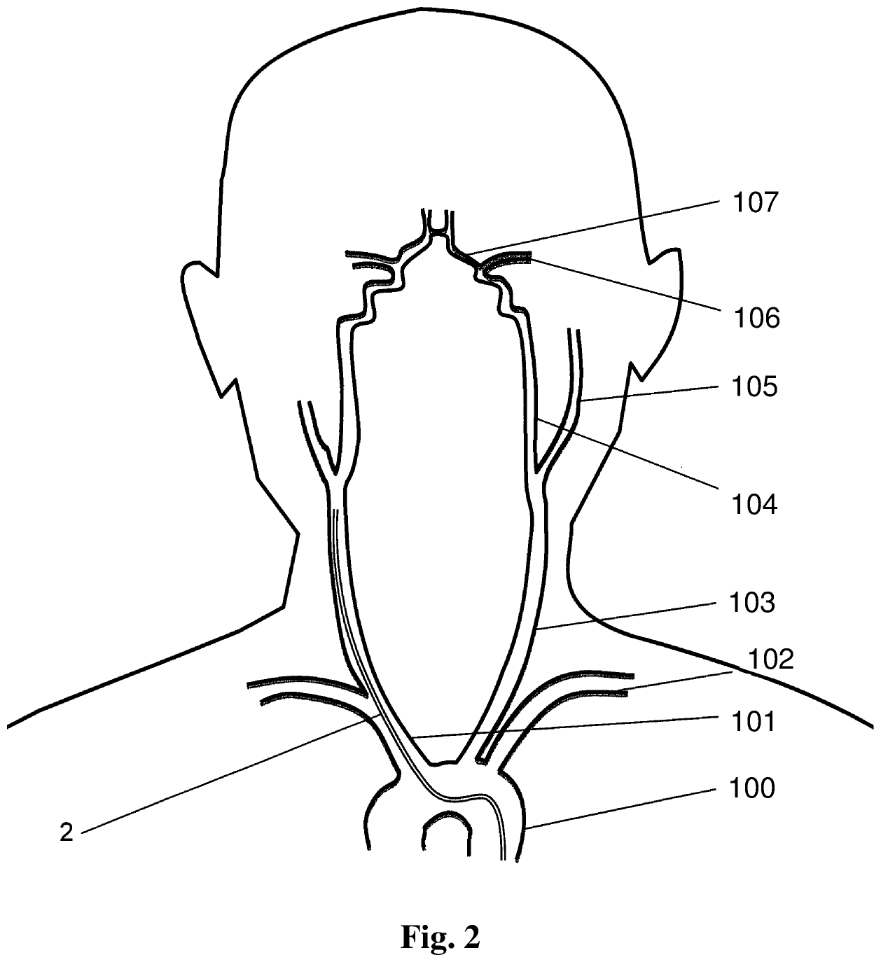

FIG. 2 shows a schematic representation of certain example cerebral vessels. Vessel 100 is the Aorta. Vessel 101 is the brachiocephalic artery. Vessel 102 is the subclavian artery. Vessel 103 is the common carotid artery. Vessel 104 is the internal carotid artery. Vessel 105 is the external carotid artery. Vessel 106 is the middle cerebral artery. Vessel 107 is the anterio-cerebral artery. The catheter 2 from FIG. 1 is shown with its distal end in the common carotid artery. In the more detailed drawings of the invention the details of the access site will not be shown but in general access and delivery is in accordance with FIGS. 1 and 2. Device 200 can be designed for use in the anterior and posterior neurovasculature in vessels such as the internal carotid artery, the M1 and M2 segments of the middle cerebral artery, the vertebral artery, and the basilar arteries. Device 200 can be delivered endovascularly under fluoroscopic guidance in a similar manner to that of other neurovascular clot-retrieval systems.

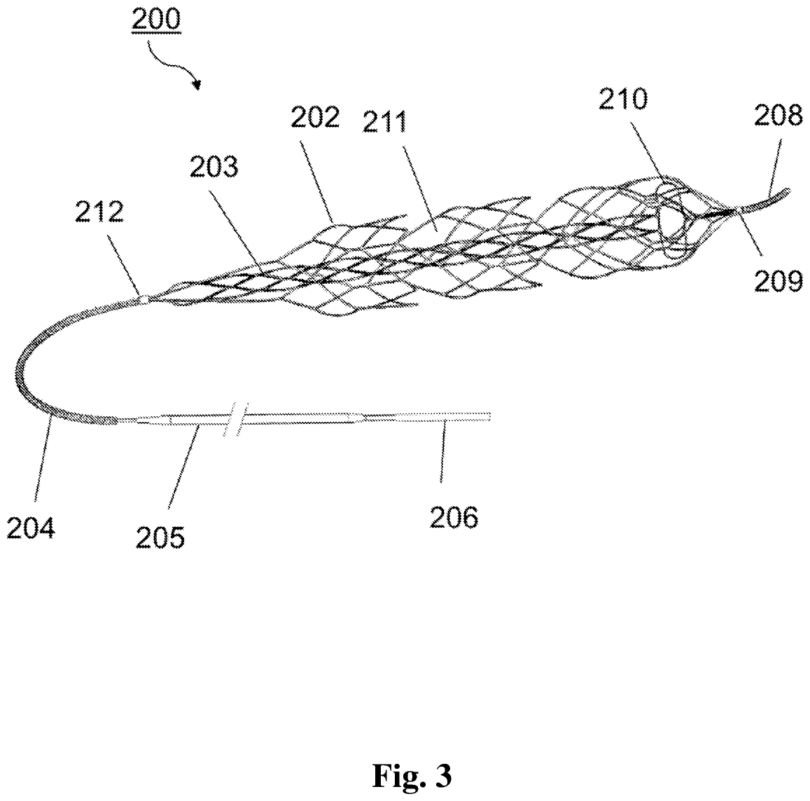

Once across the site of vessel occlusion, the stent-like element of device 200 is deployed to entrap the clot and allow it to be retrieved, hence restoring blood flow. Device 200 can be a dual-layer stent retriever, with articulating petals, and a distal capture zone for effectively trapping, retaining, and removing various clot types to restore blood flow in patients with AIS secondary to large-vessel occlusion. Examples of the device 200 can be available in two lengths, 5×21 mm and 5×33 mm. It is understood that device 200 of this disclosure would be used with a delivery system to the site of the clot, including a guide catheter, a microcatheter, and/or a guidewire. It is also contemplated that device 200 of this disclosure could be used in connection with an aspiration system to further facilitate restoring perfusion to the vasculature. FIG. 3 shows one embodiment of an example clot retrieval device of this disclosure. Device 200 can have an elongate shaft 206. Shaft 206 can have a distal end that extends interior of the artery and a proximal end that extends exterior of the artery. Shaft 206 can also have a clot engaging portion configured at its distal end having an outer expandable member 202 and an inner expandable member 203 to facilitate restoration of blood flow through the clot after device 200 is deployed. Members 202 and 203 can be configured to have a collapsed configuration for delivery and an expanded configuration for clot retrieval, restoration of perfusion, and fragmentation protection in general.

Shaft 206 may be a tapered wire shaft, and may be made of stainless steel, MP35N, Nitinol or other material of a suitably high modulus and tensile strength. Shaft 206 has a coil 204 adjacent its distal end and proximal of the outer member and inner tubular member. The coil may be coated with a low friction material or have a polymeric jacket positioned on the outer surface. Sleeve 205 may be positioned on shaft 206 adjacent coil 204. Sleeve 205 may be polymeric and may be positioned over the tapered section of shaft 206.

The outer member 202 is configured to self-expand upon release from a microcatheter to a diameter larger than that of the inner tubular member 203. Expansion of the outer member 202 causes compression and/or displacement of the clot during expansion for purposes of restoring perfusion to the vessel. A radiopaque coil 208 (which may be platinum or gold or an alloy of same) is positioned over the distal end of member 203 and butts against the distal collar 209 of the outer member 202, where it is connected by an adhesive joint to the collar 209. In some examples, the distal end of device 200 at or adjacent collar 209 can be closed by way of struts 210 being joined. In some examples, the outer member 202 can have a closed distal clot capture structure whereby a plurality of struts converge at a terminal connection. In some examples, the distal end of the outer member 202 can have its struts terminate at a distal end in a junction to define a closed end that can prevent egress of clot (or clot fragments that have entered thereof) between the inner 203 and outer 202 members. Inlet openings of outer member 202 can provide the primary movement freedom available to the clot and so the expansion of the outer member 202 urges the clot into the reception space 211 and outer member 202 can have multiple inlet mouths to accept the clot. Optionally expanded distal struts 210 can be included with the inner member 203 and function as an additional three-dimensional filter to prevent the egress of clot or clot fragments.

### Study Overview

This disclosure is more clearly understood with two corresponding studies discussed more particularly below with respect to treatment of acute ischemic stroke: (1) Rai A T, Crivera C, Kalsekar I, Kumari R, Patino N, Chekani F, and Khanna R. Endovascular Stroke Therapy Trends From 2011 to 2017 Show Significant Improvement in Clinical and Economic Outcomes. Stroke 2019; 50:1902-06; and (2) Rai A T, Crivera C, Kottenmeier E, Kalsekar I, Kumari R, Patino N, Chekani F, and Khanna R. Outcomes associated with endovascular treatment among patients with acute ischemic stroke in the USA. J. Neurolntervent. Surg. 2020; 12:422-26, each incorporated by reference in their entirety into this application as if set forth in full and attached in the appendix to priority application U.S. 63/225,245. It is understood that data is presented herein for purposes of illustration and should not be construed as limiting the scope of the disclosed technology in any way or excluding any alternative or additional embodiments.

A primary objective of the studies of this disclosure were to compare real-world primary outcomes, and economic costs among patients undergoing mechanical thrombectomy procedures for treatment of acute ischemic stroke with revascularization or clot retrieval device 200, which can include the EmboTrap retrieval device (EmboTrap, CERENOVUS, Miami, Fla., USA), and can include an open outer cage designed for clot capture and a closed inner channel designed for clot stabilization, as shown in U.S. Pat. Nos. 8,777,976; 8,852,205; 9,402,707; 9,445,829; and 9,642,639.

Initial prospective, multi-center studies using device 200 reported high reperfusion rates (mTICI≥2b within 3 passes of device 200 without rescue) of 75.0% and 80.2%, respectively. See Zaidat O O, Bozorgchami H, Ribo M, et al. Primary Results of the Multicenter ARISE II Study (Analysis of Revascularization in Ischemic Stroke With EmboTrap). Stroke 2018; 49:1107-15; and Mattle H P, Scarrott C, Claffey M, et al. Analysis of revascularisation in ischaemic stroke with EmboTrap (ARISE I study) and meta-analysis of thrombectomy. Intery Neuroradiol 2019; 25:261-70. These initial prospective studies had prespecified patient inclusion and exclusion criteria, and thus patient outcomes may differ from real-world use of device 200.

Though previous randomized clinical trials have demonstrated that mechanical thrombectomy (MT) using stent retrievers is a safe and effective treatment for acute ischemic stroke (AIS) due to large vessel occlusion (LVO), several studies using device 200 in a real-world setting have been limited to either revascularization rates and functional independence. See Brouwer P A, Yeo L L L, Holmberg A, et al. Thrombectomy using the EmboTrap device: core laboratory-assessed results in 201 consecutive patients in a real-world setting. J Neurointery Surg 2018; 10:964-68; Bourcier R, Abed D, Piotin M, et al. Multicenter initial experience with the EmboTrap device in acute anterior ischemic stroke. J Neuroradiol 2018; 45:230-35; and Valente I, Nappini S, Renieri L, et al. Initial experience with the novel EmboTrap II clot-retrieving device for the treatment of ischaemic stroke. Intery Neuroradiol 2019; 25:271-76. These previous studies did not examine the real-world hospital resource utilization, economic costs, and mortality associated with the use of the device 200 in patients with acute ischemic stroke from a retrospective analysis.

To resolve the limitation, these studies utilized a nationally representative hospital billing dataset to perform retrospective cohort analyses comparing hospital resource utilization, economic costs, and mortality in terms of real-world clinical and economic outcomes. In particular, the studies included retrospective cohort analyses of hospital-level data from the Premier Healthcare Database (PHD) for patients who underwent mechanical thrombectomy procedures for acute ischemic stroke treatment using device 200 between July 2018 and December 2019 and compared those results to two other thrombectomy devices studies using any device between 2011-2017. Data from the PHD used in these studies contained clinical coding, hospital cost, and patient billing data from more than 1000 hospitals throughout the United States. Although the database excluded federally funded hospitals (e.g., Veterans Affairs), the hospitals included were nationally representative based on bed size, geographic region, location (urban/rural), and teaching hospital status. The database contained a date-stamped log of all billed items by cost-accounting department including medications; laboratory, diagnostic, and therapeutic services; and primary and secondary diagnoses for each patient's hospitalization. Identifier-linked enrollment files provided demographic and payor information. Detailed service-level information for each hospital day was recorded, including details about medications and devices. The Premier health care alliance was formed for hospitals to share knowledge, improve patient safety, and reduce risks.

For both studies, patient characteristics including age group (18-49, 50-59, 60-69, 70+), sex, race, payor type (commercial, Medicare or Medicaid, other), existing comorbidities, Elixhauser comorbidity index (score 0-1; score 2-3; score 4+), and tPA use during index admission were collected. See Elixhauser A, Steiner C, Harris D R, et al. Comorbidity measures for use with administrative data. Med Care 1998; 36:8-27. Hospital characteristics were also collected, including teaching status (teaching/non-teaching), geographic location (Midwest, Northeast, South, West), size (<300 beds, 300-499 beds, and ≥500 beds), and urban-rural classification.

This data was collected for the date of index hospitalization. Comorbidities were assessed using Charlson Comorbidity Index (CCI) scores. de Groot V, Beckerman H, Lankhorst G J, et al. How to measure comorbidity: a critical review of available methods. J Clin Epidemiol 2003; 56:221-29. doi: 10.1016/S0895-4356(02)00585-1. The CCI is understood as an aggregate measure of comorbidity that combines 19 select diagnoses associated with chronic disease (e.g., heart disease, cancer) weighted on a scale from +1 to +6. Higher scores were indicative of greater comorbidity burden. Data was also controlled for specific comorbidities including diabetes, hypertension, polycystic kidney disease, congestive heart failure, peripheral vascular disorder, chronic pulmonary disease, ischemic stroke/transient ischemic attack (TIA), hypothyroidism, obesity, and depression.

Hospital characteristics were also included in the data, including teaching status (teaching/non-teaching), geographic location (Midwest, Northeast, South, West), size (<300 beds, 300-499 beds, and ≥500 beds), urban-rural classification, and volume of mechanical thrombectomy procedures for AIS in the 12-month pre-index period. With this data, the studies analyzed primary outcomes, such as discharge status (including in-hospital mortality, discharge to home/home health organization, discharge to skilled nursing facility, and others [including discharge/transfer to other facility, discharge to rehab facility, discharge to hospice-medical facility, etc.]), intracranial hemorrhage (ICH), mean length of stay (LOS), mean hospital cost, and 30-day readmissions (all-cause, cardiovascular [CV]-related, and AIS-related) The 12-month total inpatient costs (sum of index admission cost and 12-month inpatient readmission cost) was also assessed. Readmissions is a key marker of long-term treatment effectiveness when considering secondary data sources such as the PHD, which do not include tailored study endpoint variables.

Mean hospital costs (reported in 2020 USD) capture all billable items including room and board, supply, pharmacy, and laboratory costs. Inpatient readmissions (all-cause, CV-related, and AIS-related) were assessed among patients who were treated in a hospital that continuously provided data to the PHD for 30 days post-procedure. All costs were adjusted for medical inflation and are reported in 2020 US dollars ($). As healthcare dollars are increasingly scrutinized, it is important to understand cost differences associated with device selection from both a payer and provider perspective.

All study variables are summarized as the mean and standard deviation for continuous variables and the frequency and percentage for categorical variables. Bivariate statistical tests (Chi square tests for categorical variables and t-tests for continuous variable comparison) were conducted to examine and describe between-group differences in potential confounding factors such as patient demographics, clinical characteristics, procedural characteristics, and provider characteristics. Generalized estimating equation (GEE) models with an exchangeable correlation structure and appropriate link (logit link for discharge status, complications, and readmission comparison; log link for LOS and cost comparison) and distribution function (binomial distribution for discharge status, complications, and readmission; negative binomial distribution for LOS; gamma distribution for cost) were utilized to compare outcomes between the studies. GEE analyses adjusted comparisons for hospital clustering and any covariate that emerged significant post-matching (i.e., SMD≥0.10 or ≤−0.10). Outcomes investigated included length of stay (LOS), discharge status (mortality), 12-month inpatient readmission, and total cost of care (index admission plus 12-month all-cause readmission).

For hospitals treating stroke patients, including acute ischemic stroke, the 30-day readmission rate is a key indicator of quality of care and is increasingly tied to value-based reimbursement. As such, it is important to understand this outcome among patients treated with novel MT devices such as device 200. Studies have reported considerable variation in 30-day readmission rates among AIS patients, ranging from 6.0% to 15.0%. See, e.g., Bjerkreim A T, Khanevski A N, Selvik H A, et al. The Impact of Ischaemic Stroke Subtype on 30-day Hospital Readmissions. Stroke Res Treat 2018; 2018:7195369; Nouh A M, McCormick L, Modak J, et al. High Mortality among 30-Day Readmission after Stroke: Predictors and Etiologies of Readmission. Front Neurol 2017; 8:632. This variation may be due to differences in readmission definitions across studies, including whether or not studies included elective readmissions, distinguished between all-cause and specific diagnosis-related readmissions, or if specific patient populations were excluded from readmission counts due to their discharge category. In contrast, the current study included both elective and non-elective procedures when determining the rate of readmission. These differences make it difficult to compare readmission rates from the first study of this disclosure with those from prior studies.

### First Study Population

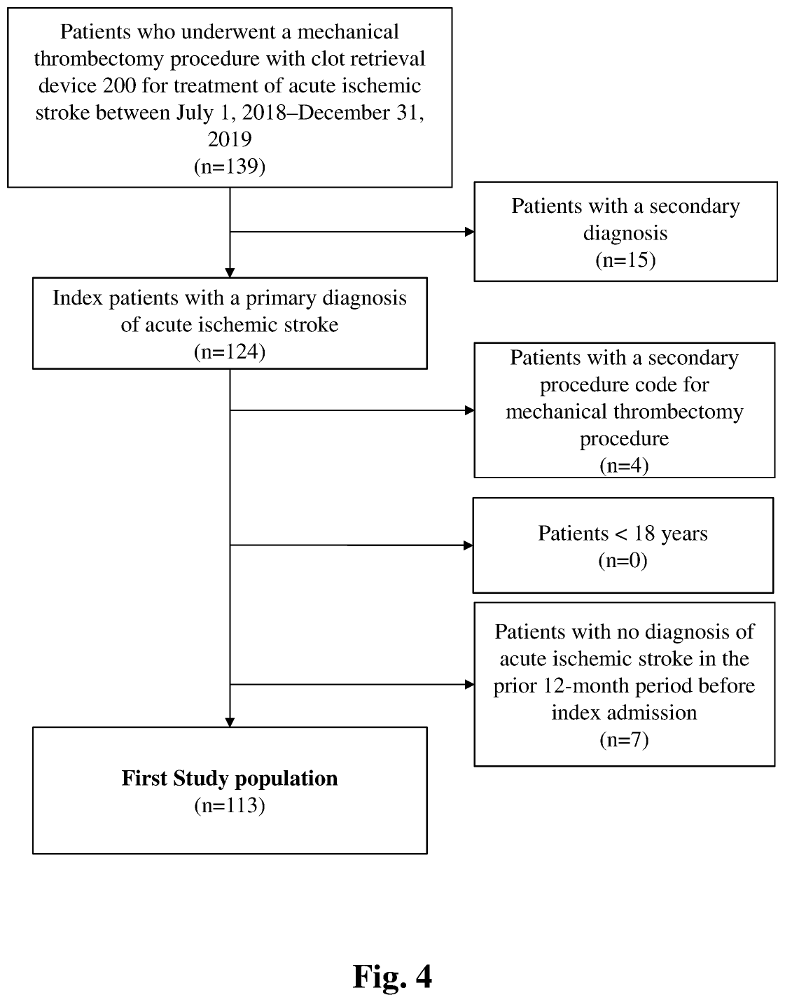

The first study cohort attrition process is shown in FIG. 4. The first study population included patients aged 18 years or older with a first primary diagnosis of acute ischemic stroke (International Classification of Disease, 9th edition, clinical modification codes for Acute Ischemic Stroke (ICD-9-CM): 433.xx; 434.xx; 436.xx, 437.xx, 438.xx and equivalent ICD-10-CM code), who underwent mechanical thrombectomy procedure with clot retrieval device 200 between July 2018 and December 2019 as identified from the PHD. Eligible patients had a primary or secondary procedure code for mechanical thrombectomy during an inpatient stay. Patients were excluded if they had a primary or secondary diagnosis of acute ischemic stroke in any setting in the PHD in the 12-month period prior to index admission.

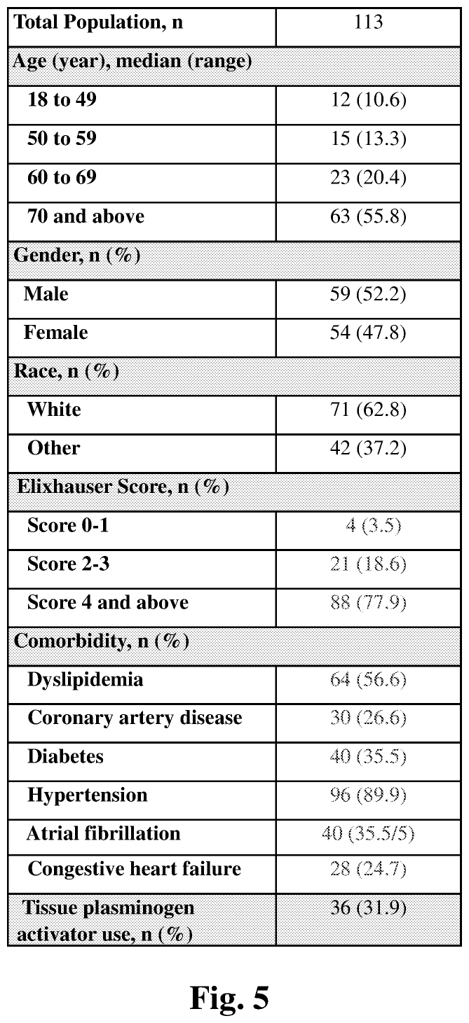

FIG. 5 shows a table summarizing patient demographics and characteristics for the first study. Of the 113 patients identified in the study that were treated with device 200, over half the patients were age ≥70 years (55.8%; 63/113; mean age 69.1±13.5 years). Additionally, over half of the patients were male (52.2%; 59/113) and white (62.8%; 71/113). The majority (81.4%; 92/113) of patients were enrolled in Medicare or Medicaid, with 14.2% (16/113) having commercial coverage. A total of 77.8% (88/113) had an Elixhauser Score of 4 or higher. Patient comorbidities included diabetes (35.5%; 40/113), hypertension (84.9%; 96/113), atrial fibrillation (35.5%; 40/113), and congestive heart failure (24.7%; 28/113). Dyslipidemia was reported in over half of the patients (56.6%; 64/113), and coronary artery disease in 26.6% (30/113). Nearly one third of patients (31.9%; 36/113) were treated with tPA.

Geographically, 63.7% (72/113) of the cases were reported by hospitals in the South, with a high concentration in urban areas (91.2%; 103/113). Many were teaching hospitals (83.2%; 94/113), and 86.7% (98/113) were large hospitals with 500 beds or more.

### Results of First Study

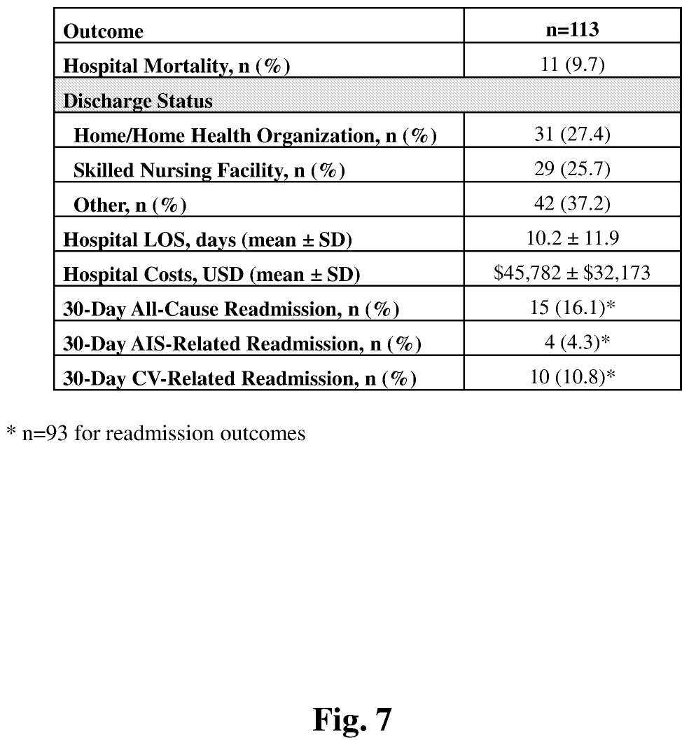

First study outcomes for patients treated using device 200 are detailed in FIG. 7. The overall in-hospital mortality rate was 9.7% (11/113). Among those discharged, 27.4% (31/113) were discharged to home/home health organization, 25.7% (29/113) were discharged to a skilled nursing facility, and the remaining 37.2% (42/113) were discharged to another type of facility such as a rehab facility, or hospice-medical facility. The rate of ICH among patients treated with device 200 was 17.7% (20/113). The mean LOS among patients undergoing MT using device 200 was 10.2±11.9 days (median 6.0 days). The mean hospital costs were $45,782±$32,173 (median $37,076). The 30-day all-cause, CV-related, and AIS-related inpatient readmission rates were 16.1% (15/93), 10.8% (10/93), and 4.3% (4/93), respectively, among patients who had a procedure in hospitals that continuously provided data to the PHD for 30 days post procedure.

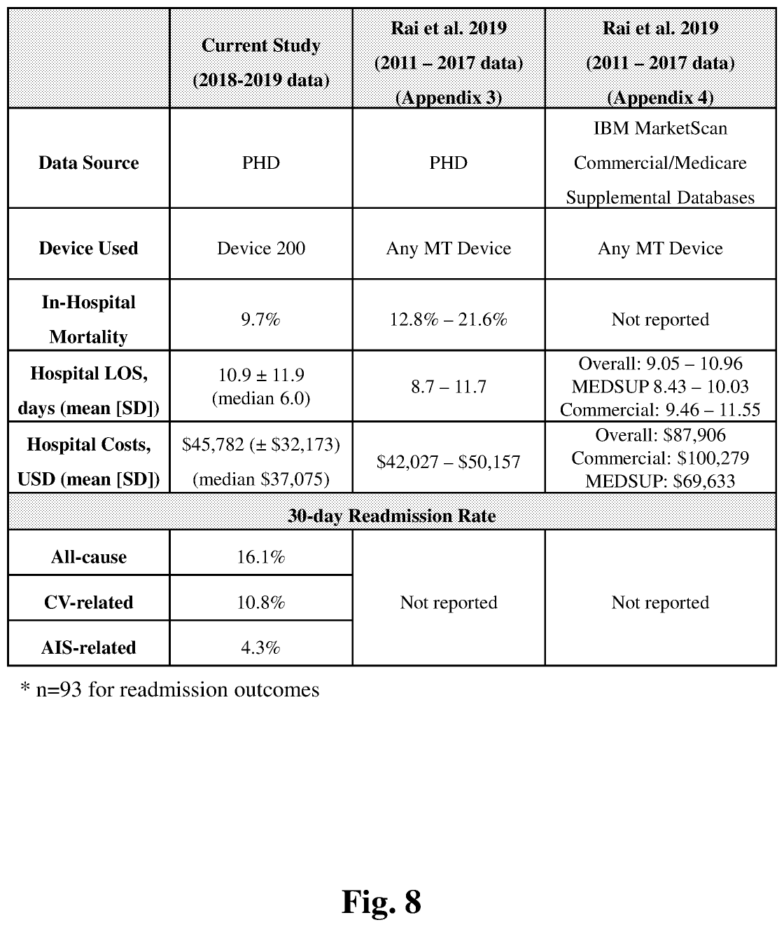

Compared to the prospective studies assessing hospital outcomes among patients undergoing endovascular treatment for acute ischemic stroke using two other thrombectomy devices between 2011 and 2017, the reported in-hospital mortality for those patients treated with device 200 is reduced, while hospital LOS and costs remain the same. The comparison of results of the first study and two other real-world studies is described in FIG. 8. In particular, using the same retrospective claim database and comparing results from any endovascular thrombectomy device to that of exclusively device 200, the in-hospital mortality rates for patients undergoing endovascular treatment using any mechanical thrombectomy device ranged from 12.8% to 21.6%, which is higher than the rate of 9.7% reported for patients undergoing endovascular treatment using device 200. The difference ranges from about 3.1% to about 11.9% may be attributable to the use of device 200.

Costs in the first study using device 200 were similar to index admission costs reported by the other two trials, which ranged from $42,027-$50,157.20 for patients treated with the other revascularization devices and tPA and approximately $45,761 for patients for patients treated with device 200.

The 30-day all-cause readmission rate for patients treated using device 200 was 16.1%, while the cardiovascular-related and acute ischemic stroke-related readmission rates were 10.8% and 4.3%, respectively.

### Second Study Population

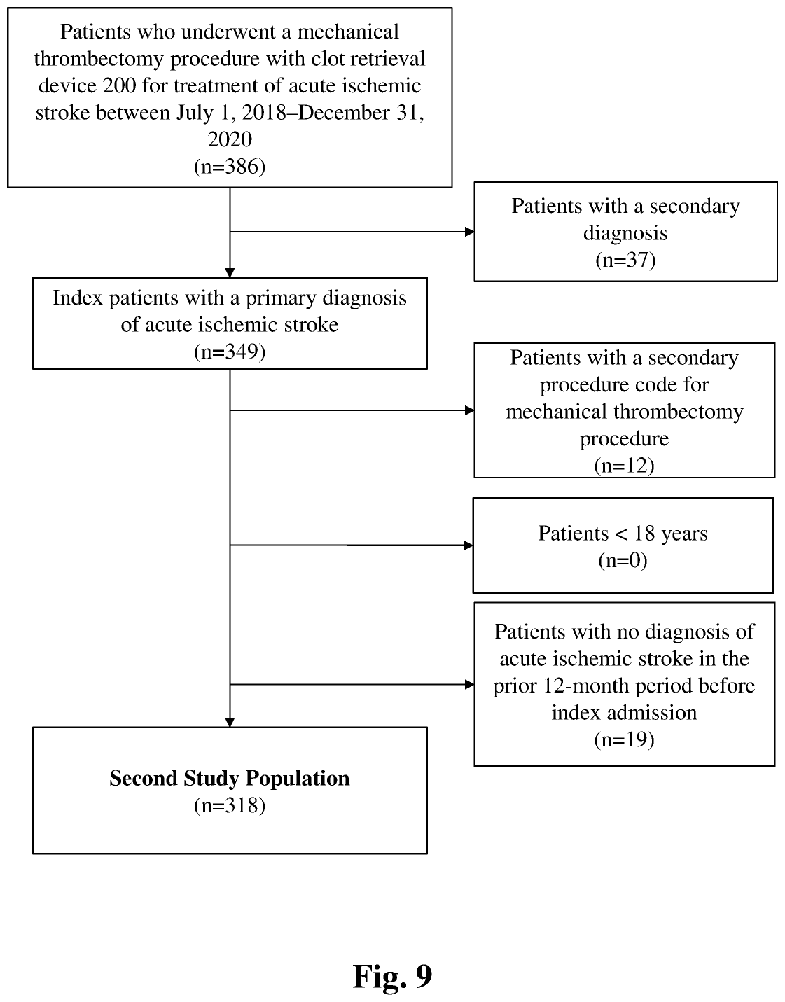

The second study cohort attrition process is shown in FIG. 9. The second study population included all those of the first study population with the inclusion of patients who underwent a mechanical thrombectomy procedure with clot retrieval device 200 for treatment of acute ischemic stroke between Jul. 1, 2018 and Dec. 31, 2020, as identified from the PHD. Eligible patients had a primary or secondary procedure code for mechanical thrombectomy during an inpatient stay. Patients were excluded if they had a primary or secondary diagnosis of acute ischemic stroke in any setting in the PHD in the 12-month period prior to index admission.

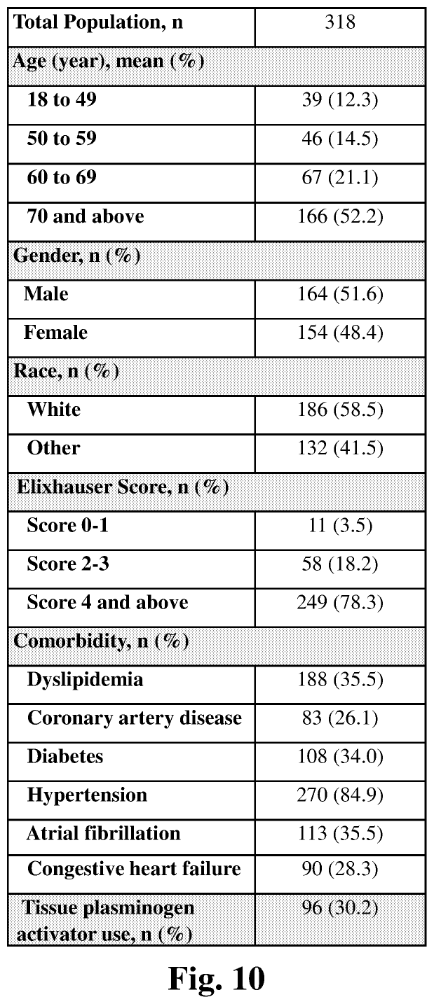

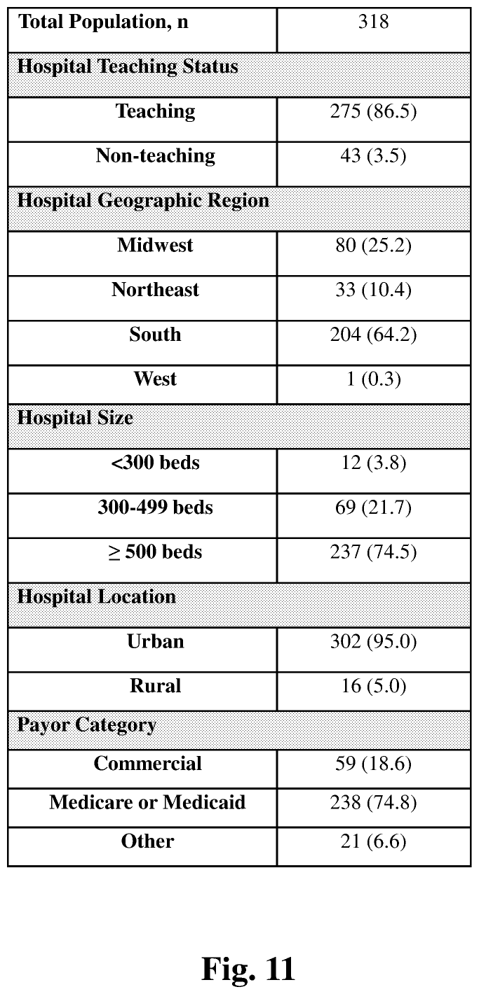

FIGS. 10 and 11 show tables summarizing patient demographics and characteristics for the second study. Of the 318 patients identified in the study that were treated with device 200, over half the patients were age ≥70 years (52.2%%; 166/318). Additionally, just over half of the patients were male (51.6%; 164/318) and white (58.5%; 164/318). The majority (74.8%; 238/318) of patients were enrolled in Medicare or Medicaid, with 18.6% (59/318) having commercial coverage. A total of 78.3% (249/318) had an Elixhauser Score of 4 or higher. Patient comorbidities included diabetes (34.0%; 108/318), hypertension (84.9%; 270/318), atrial fibrillation (35.5%; 113/318), and congestive heart failure (28.3%; 90/318). Differing from the first study. dyslipidemia was identified in less than half of the patients (35.5%; 188/318), and coronary artery disease in 26.1% (83/318). Nearly one third of patients (30.2%; 96/318) were treated with tPA.

Geographically, 64.2% (204/318) of the cases were reported by hospitals in the South, with a high concentration in urban areas (95.0%; 302/318). Many were teaching hospitals (86.5%; 275/318), and 74.5% (237/318) were large hospitals with 500 beds or more.

### Results of Second Study

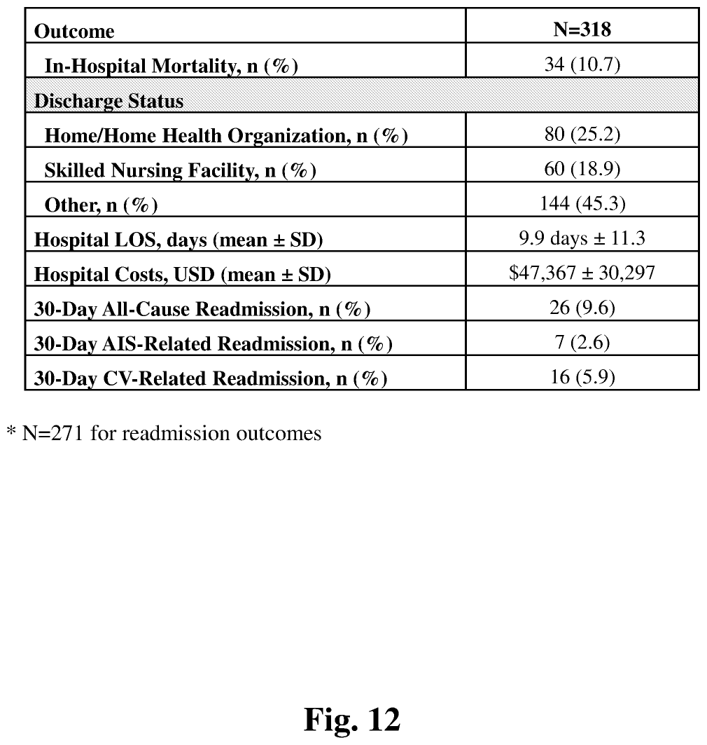

Second study outcomes for patients treated using device 200 are detailed in FIG. 12. The overall in-hospital mortality rate was 10.7% (34/318). Among those discharged, 25.2% (80/318) were discharged to home/home health organization, 18.9% (60/318) were discharged to a skilled nursing facility, and the remaining 45.3% (144/318) were discharged to another type of facility such as a rehab facility, or hospice-medical facility. The mean LOS among patients undergoing MT using device 200 was 9.9±11.3 days. The mean hospital costs were $47,367±$30,297. The 30-day all-cause, CV-related, and AIS-related inpatient readmission rates were 9.6% (26/318), 2.6% (7/318), and 5.9% (16/318), respectively, among patients who had a procedure in hospitals that continuously provided data to the PHD for 30 days post procedure.

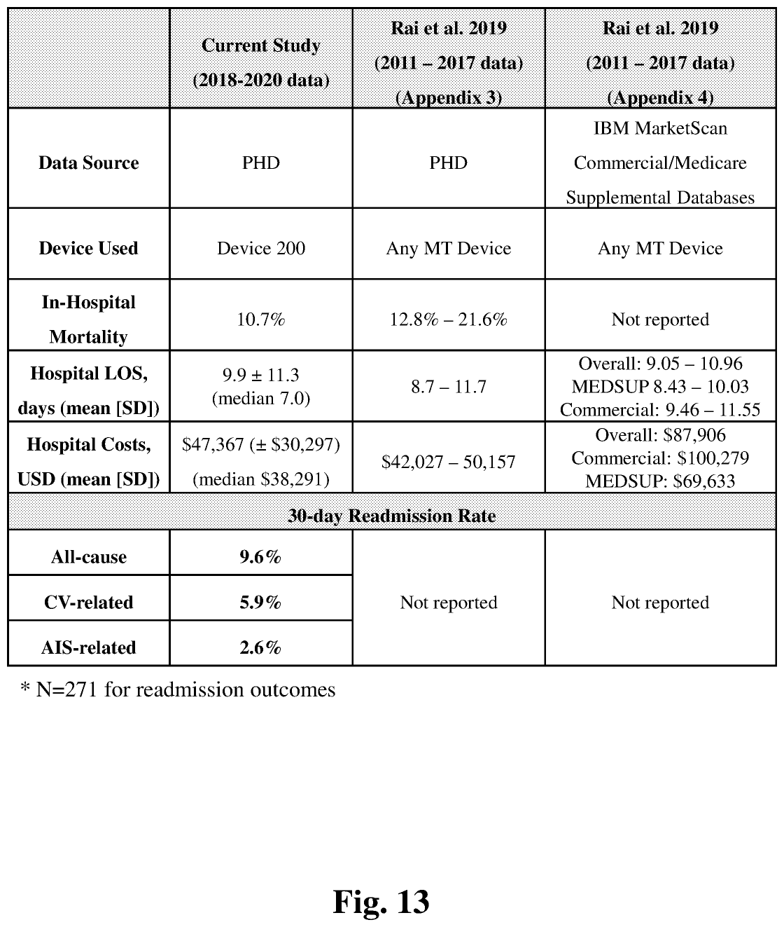

Compared to the prospective studies assessing hospital outcomes among patients undergoing endovascular treatment for acute ischemic stroke using two other thrombectomy devices between 2011 and 2017, the reported in-hospital mortality for those patients treated with device 200 is reduced, while hospital LOS and costs remain the same. The comparison of results of the second study and two other real-world studies is described in FIG. 13. In particular, using the same retrospective claim database and comparing results from any endovascular thrombectomy device to that of exclusively device 200, the in-hospital mortality rates for patients undergoing endovascular treatment using any mechanical thrombectomy device ranged from 12.8% to 21.6%, which is higher than the rate of 10.7% reported for patients undergoing endovascular treatment using device 200 between 2018 and 2020. The difference ranges from about 2.1% to about 10.9% may be attributable to the use of device 200.

Costs in the second study using device 200 were similar to index admission costs reported by the other two trials, which ranged from $42,027-$50,157.20 for patients treated with the other revascularization devices and tPA and about $47,367 for patients treated with device 200.

The 30-day all-cause readmission rate for patients treated using device 200 was 9.6%, while the cardiovascular-related and acute ischemic stroke-related readmission rates were 5.9% and 2.6%, respectively.

### Third Study Population

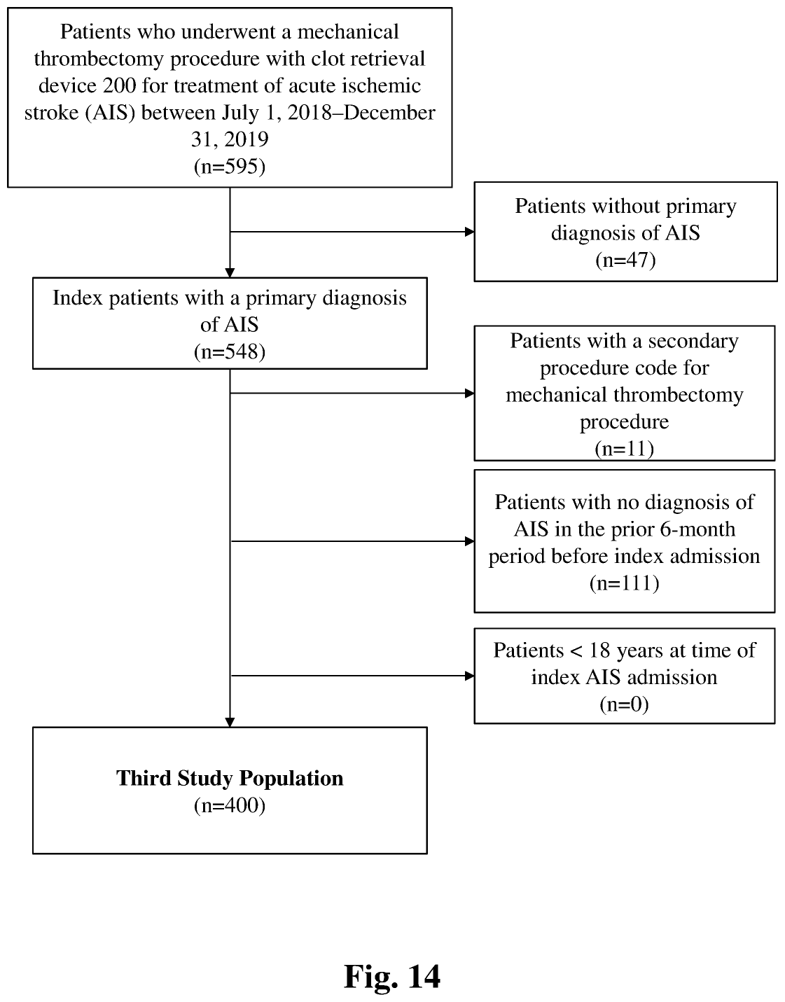

The third study cohort attrition process is shown in FIG. 14. The third study population included patients aged 18 years or older with a first primary diagnosis of acute ischemic stroke (International Classification of Disease, 9th edition, clinical modification codes for Acute Ischemic Stroke (ICD-9-CM): 433.xx; 434.xx; 436.xx, 437.xx, 438.xx and equivalent ICD-10-CM code), who underwent mechanical thrombectomy procedure with clot retrieval device 200 between July 2018 and December 2019 as identified from the PHD. Eligible patients had a primary or secondary procedure code for mechanical thrombectomy during an inpatient stay. Patients were excluded if they had a primary or secondary diagnosis of acute ischemic stroke in any setting in the PHD in the 12-month period prior to index admission.

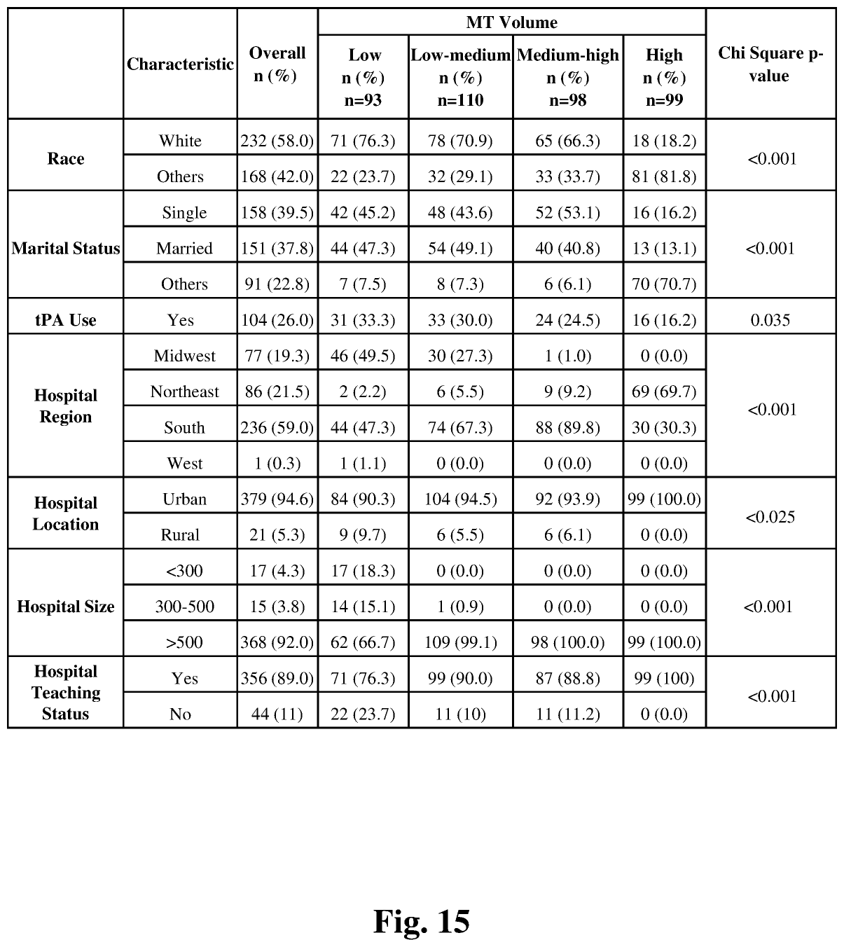

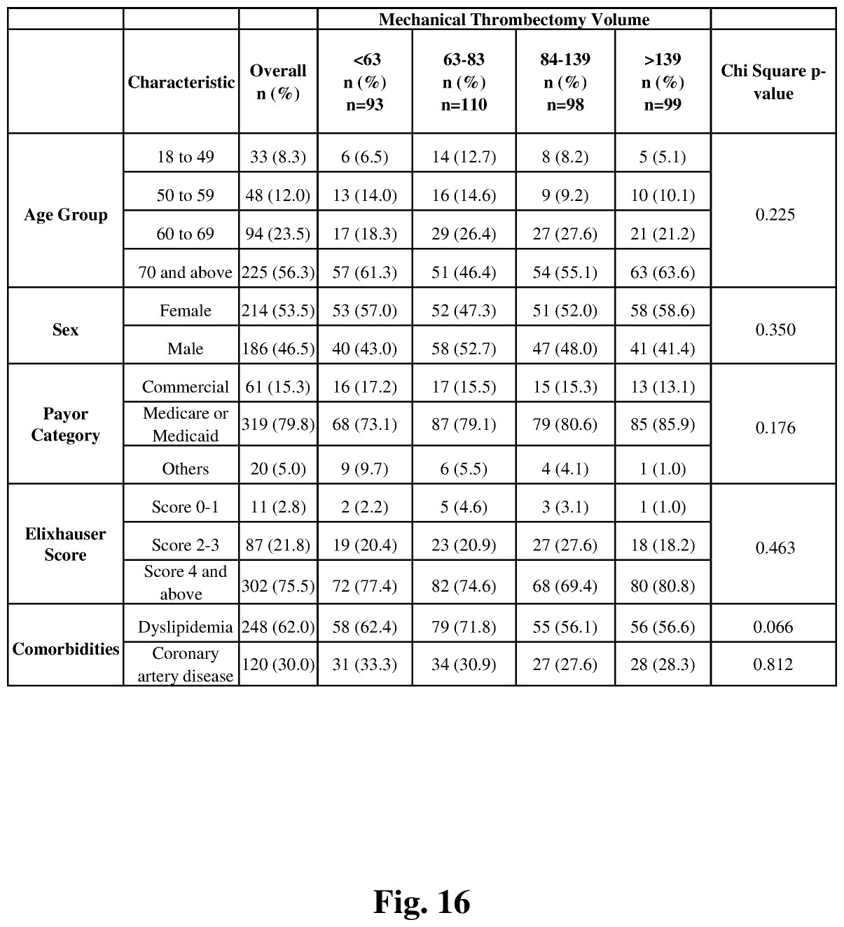

FIGS. 15 and 16 show table summarizing patient demographics and hospital characteristics by mechanical thrombectomy volume for the third study. Of the 400 patients identified in the study that were treated with device 200, over half the patients were age ≥70 years (56.3%; 225/400; p=0.225). Unlike the first study, less than half of the patients were male (46.5%; 186/400). The majority of patients were white (58%; 232/400) and enrolled in Medicare or Medicaid (79.8%; 319/400), with 15.3% (61/400) having commercial coverage. A total of 75.5 (302/400) had an Elixhauser Score of 4 or higher. Patient comorbidities included dyslipidemia (62.0%; 248/400) and coronary artery disease (30.0%; 120/400). Some patients (26.0%; 104/400) were treated with tPA.

Geographically, over half of the cases were reported by hospitals in the South (59.0%; 236/400), with a high concentration in urban areas (94.6%; 379/400). Many were teaching hospitals (89.0%; 356/400), and 92.0% (368/400) were large hospitals with 500 beds or more.

### Results of Third Study

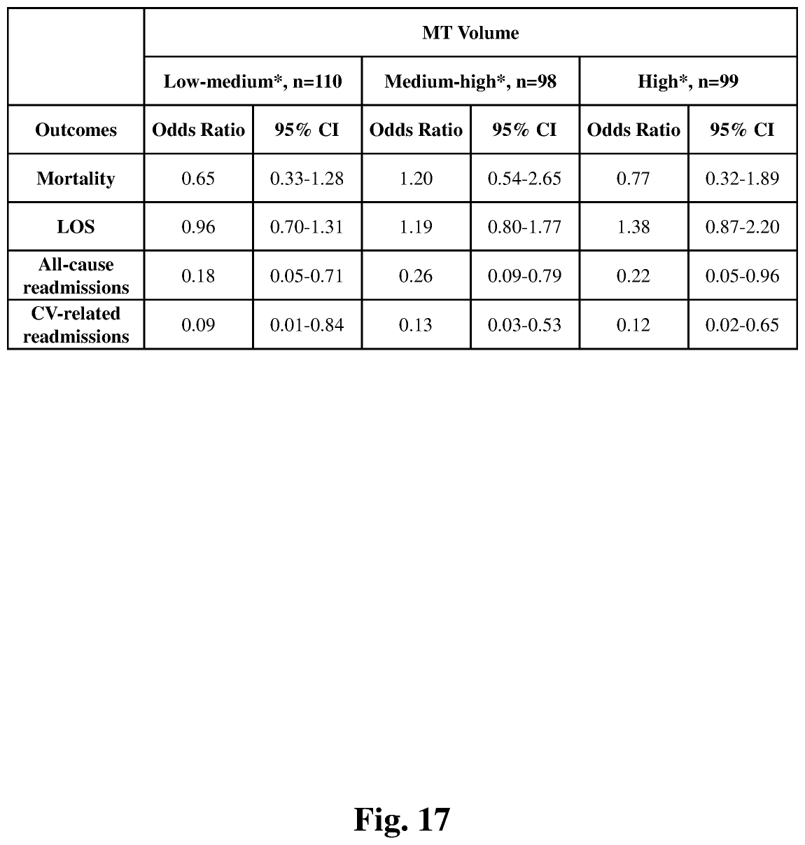

Third study outcomes for patients treated using device 200 by hospital mechanical thrombectomy (MT) volume are detailed in FIG. 17. Hospital MT volume significantly influenced the rate of readmissions among acute ischemic stroke patients treated with device 200. For a low to medium MT volume hospital, the odds ratio of in-hospital mortality rate was 0.65 (95% Confidence Interval (“CI”) 0.33-1.28), for a medium to high MT volume hospital was 1.20 (95% CI=0.54-2.65), and for a high MT volume hospital was 0.77 (95% CI=0.32-1.89). The odds ratio of LOS among patients undergoing MT using device 200 was 0.96 (95% CI=0.70-1.31) at a low to medium MT volume hospital, 1.19 (95% CI=0.80-1.77) at a medium to high MT volume hospital, and 1.38 (95% CI=0.87-2.20) at a high MT volume hospital. The odds ratio of all-cause readmission of patients undergoing MT using device 200 at a low to medium MT volume hospital was 0.18 (95% CI=0.05-0.71), for a medium-to high MT volume hospital was 0.26 (95% CI=0.09-0.79), and for a high MT volume hospital was 0.22 (95% CI=0.05-0.96). The odds ratio of cardiovascular readmission among patients undergoing MT using device 200 was 0.09 (95% CI=0.01-0.84) at a low to medium MT volume hospital, 0.13 (95% CI=0.03-0.53) at a medium to high MT volume hospital, and 0.12 (95% CI=0.02-0.65) at a high MT volume hospital.

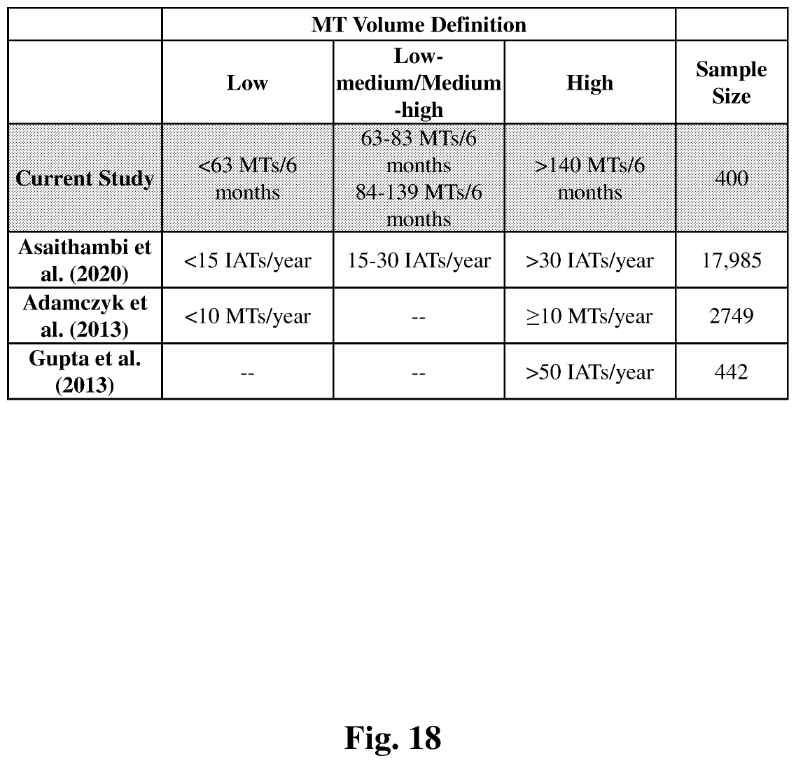

As depicted in FIG. 18, previous studies have shown that hospital MT volume may influence outcomes among patients with acute ischemic stroke that are treated with a MT device. Of those MT devices, device 200 also contributed to the significantly lower readmission rates in hospitals with higher MT volume.

In each study, device 200 was prepared for delivery to the occlusion site with standard interventional techniques to access the arterial system and using angiography in order to determine the location of the occluded vessel. Once determined, a guide catheter, sheath, or balloon guide catheter was advanced as close to the occlusion as possible. A rotating hemostasis valve (RHV) was connected to the proximal end of the catheter and connected to a continuous flush system. An appropriate microcatheter was then selected and an RHV was connected to the proximal end of the microcatheter and connected to a continuous flush system. With the aid of a suitable guidewire, and using standard catheterization techniques and fluoroscopic guidance, the microcatheter was advanced up to and across the occlusion so that the distal end of the microcatheter is positioned distal of the occlusion. The guidewire was removed from the microcatheter and optionally contrast media was gently infused through the microcatheter to visualize the distal end of the occlusion. The insertion tool with the preloaded retrieval device 200 was then removed from the packaging hoop. The distal end of the insertion tool was inserted through the RHV of the microcatheter and then waited until fluid was seen exiting the proximal end of the insertion tool, confirming that device 200 was flushed. The insertion tool was then advanced until it contacted the hub of the microcatheter and the RHV was fully tightened to hold the insertion tool securely in position. The insertion tool was confirmed as being fully seated in the hub of the RHV before proceeding to advance device 200 until at least half of the shaft length of shaft 206 was inserted into the microcatheter, at which point the insertion tool was removed.

Regarding positioning and deployment, device 200 continued to be advanced towards the distal tip of the microcatheter (e.g., until the distal radiopaque tip 208 of the device 200 was aligned with the distal tip). Device 200 optionally included bands positioned on the proximal portion of shaft 206 to assist in minimizing the amount of fluoroscopic exposure required during insertion of device 200. If using a standard microcatheter (total length of 155 cm and a 7 cm RHV), then when the first band on the shaft 206 approached the RHV, while the tip of device 200 was approximately 8 cm from the distal end of the microcatheter. When the second band on the shaft 206 approached the RHV, the tip of device 200 was nearing the distal end of the microcatheter. Device 200 was then advanced in the microcatheter and positioned within the clot and left to embed for 3-5 minutes prior to withdrawal.

Device 200 was optionally supplied preloaded within an insertion tool. In such applications, the physician inserted the insertion tool into the hub of a pre-positioned microcatheter and advances the clot retrieval device forward out of the insertion tool and into the microcatheter.

During each study, device 200 tested was typically used for up to three (3) retrieval attempts. If an additional pass was made with device 200, then any captured thrombus was carefully removed therefrom, and device 200 cleaned in heparinized saline.

Patients associated with each study included those with acute ischemic stroke in anterior circulation (including distal internal carotid artery (ICA), carotid T, middle cerebral artery (MCA) segments M1 and M2) treated with endovascular treatment using the stent retriever of this disclosure as first or second line device, were retrospectively included across multiple different centers. According to the availability of the material, there were not any recommendation regarding a preferred type of stent retriever, the clot retrieval device of this disclosure was used randomly in the flow of patients.

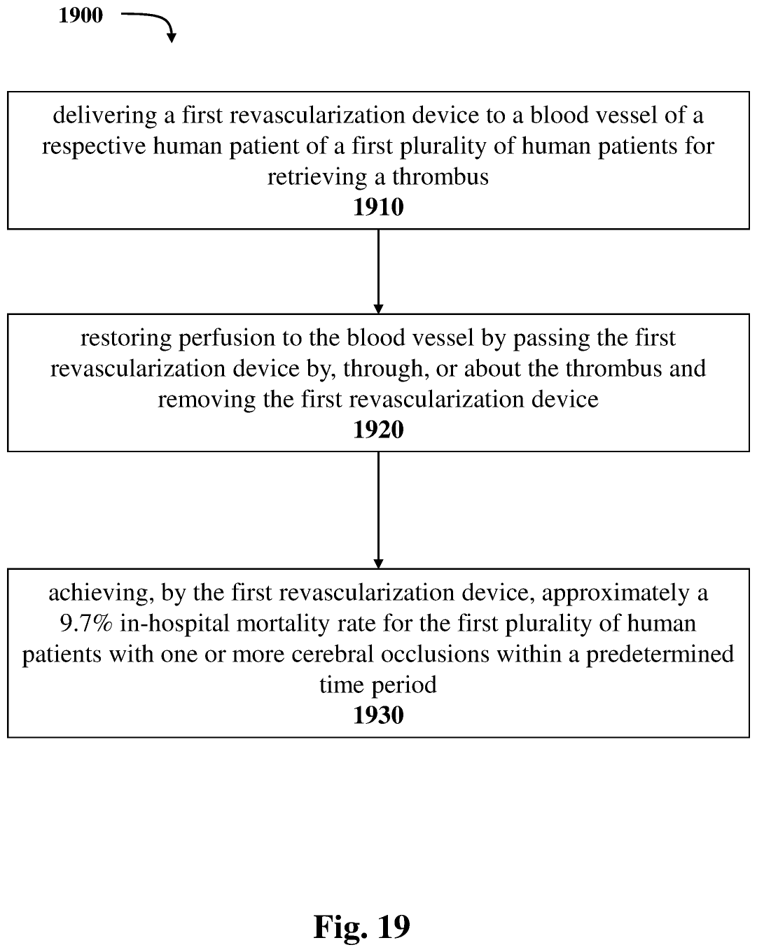

FIG. 19 depicts a method or use 1900 and can include 1910 delivering a first revascularization device to a blood vessel of a respective human patient of a first plurality of human patients for retrieving a thrombus; 1920 restoring perfusion to the blood vessel by passing the first revascularization device by, through, or about the thrombus and removing the first revascularization device; and 1930 achieving, by the first revascularization device, approximately a 9.7% in-hospital mortality rate for the first plurality of human patients with one or more cerebral occlusions within a predetermined time period. Method or use 1900 can end after step 1930.

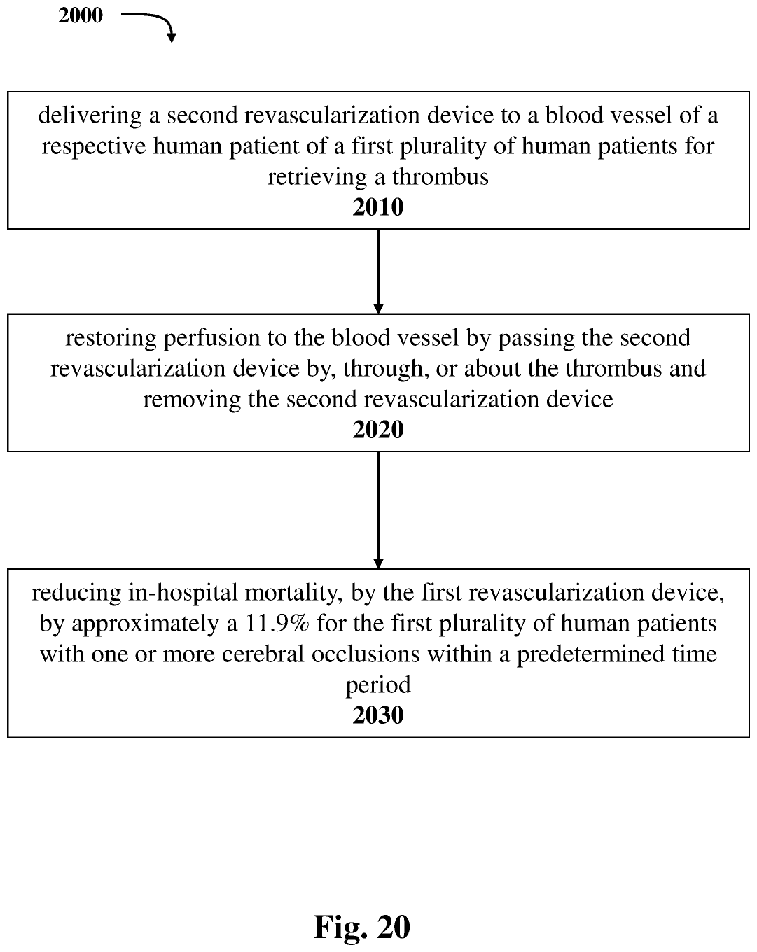

FIG. 20 depicts a method or use 2000 and can include 2010 delivering a second revascularization device to a blood vessel of a respective human patient of a first plurality of human patients for retrieving a thrombus; 2020 restoring perfusion to the blood vessel by passing the second revascularization device by, through, or about the thrombus and removing the second revascularization device; and 2030 reducing in-hospital mortality, by the first revascularization device, by approximately a 11.9% for the first plurality of human patients with one or more cerebral occlusions within a predetermined time period. Method or use 2000 can end after step 2030.

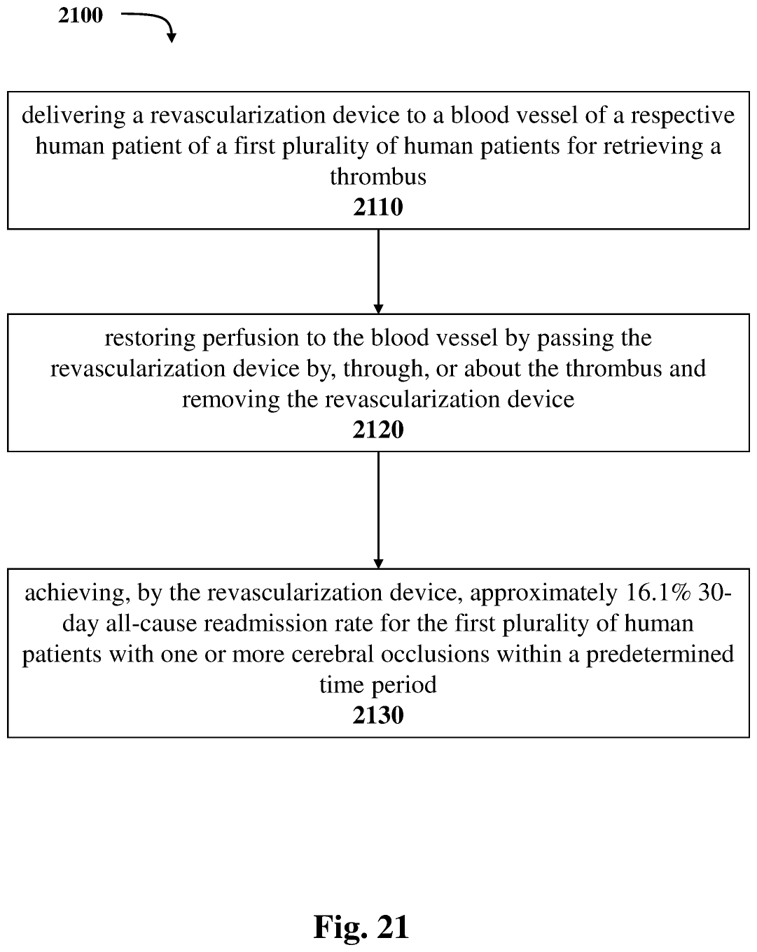

FIG. 21 depicts a method or use 2100 and can include 2110 delivering a revascularization device to a blood vessel of a respective human patient of a first plurality of human patients for retrieving a thrombus; 2120 restoring perfusion to the blood vessel by passing the revascularization device by, through, or about the thrombus and removing the revascularization device; and 2130 achieving, by the revascularization device, approximately 16.1% 30-day all-cause readmission rate for the first plurality of human patients with one or more cerebral occlusions within a predetermined time period. Method or use 2100 can end after step 2130.

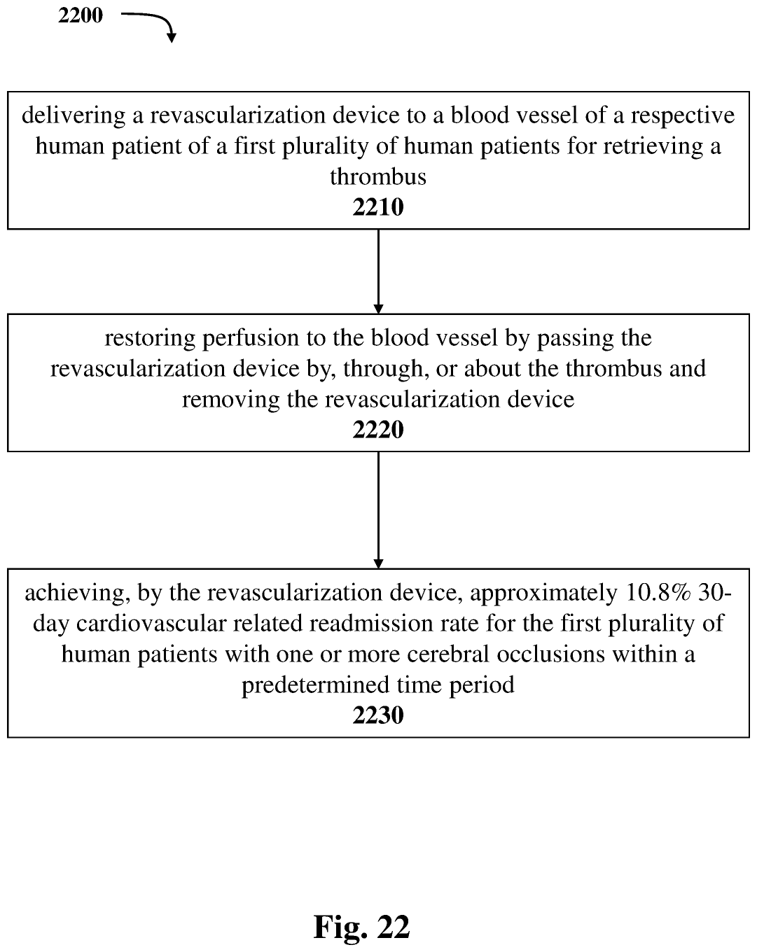

FIG. 22 depicts a method or use 2200 and can include 2210 delivering a revascularization device to a blood vessel of a respective human patient of a first plurality of human patients for retrieving a thrombus; 2220 restoring perfusion to the blood vessel by passing the revascularization device by, through, or about the thrombus and removing the revascularization device; and 2230 achieving, by the revascularization device, approximately 10.8% 30-day cardiovascular related readmission rate for the first plurality of human patients with one or more cerebral occlusions within a predetermined time period. Method or use 2200 can end after step 2230.

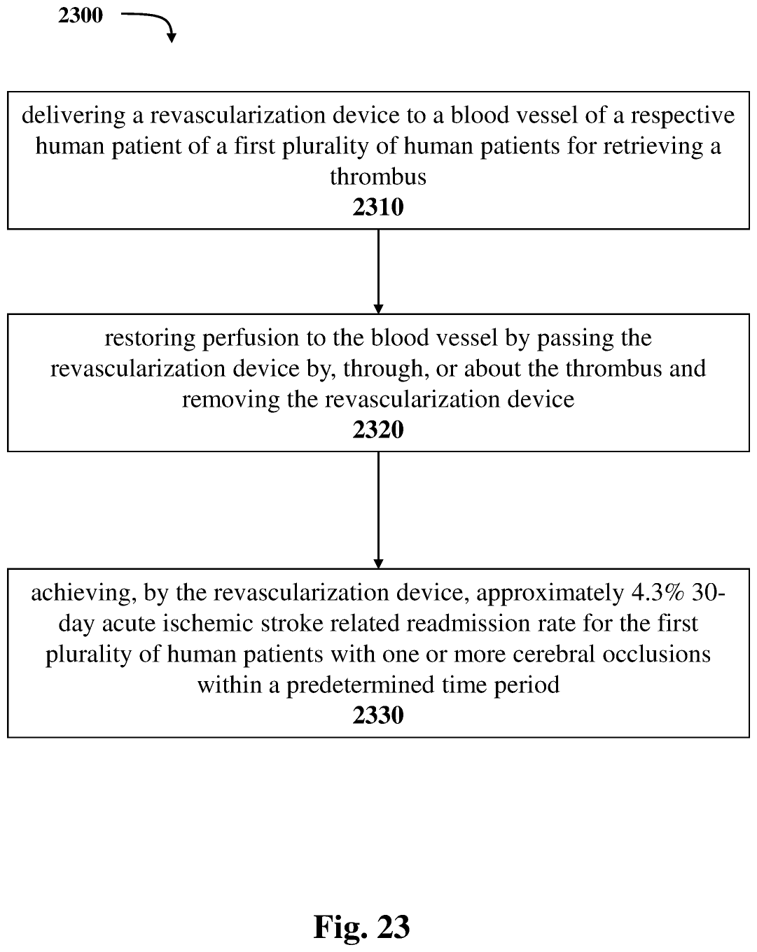

FIG. 23 depicts a method or use 2300 and can include 2310 delivering a revascularization device to a blood vessel of a respective human patient of a first plurality of human patients for retrieving a thrombus; 2320 restoring perfusion to the blood vessel by passing the revascularization device by, through, or about the thrombus and removing the revascularization device; and 2330 achieving, by the revascularization device, approximately 4.3% 30-day acute ischemic stroke related readmission rate for the first plurality of human patients with one or more cerebral occlusions within a predetermined time period. Method or use 2300 can end after step 2330.

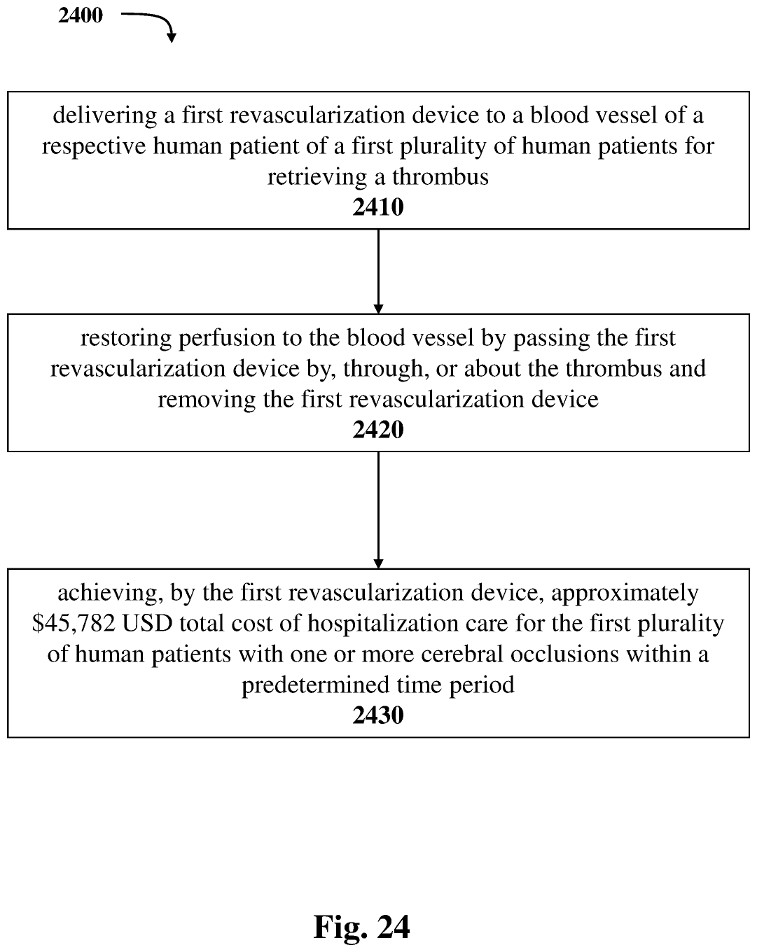

FIG. 24 depicts a method or use 2400 and can include 2410 delivering a first revascularization device to a blood vessel of a respective human patient of a first plurality of human patients for retrieving a thrombus; 2420 restoring perfusion to the blood vessel by passing the first revascularization device by, through, or about the thrombus and removing the first revascularization device; and 2430 achieving, by the first revascularization device, approximately $45,782 USD total cost of hospitalization care for the first plurality of human patients with one or more cerebral occlusions within a predetermined time period. Method or use 2400 can end after step 2430.

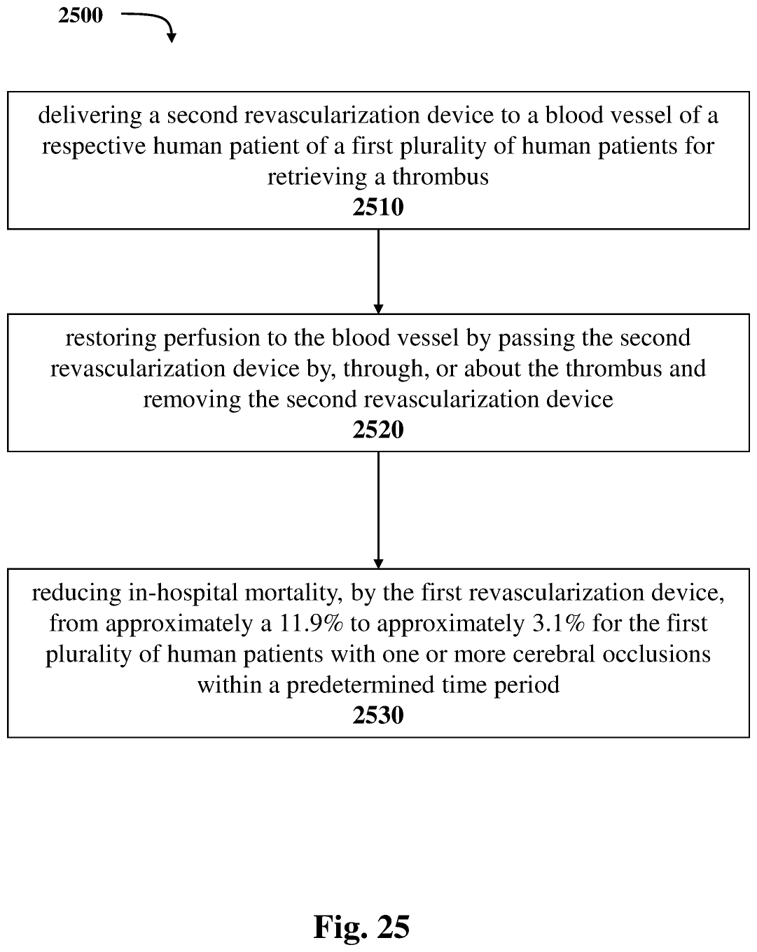

FIG. 25 depicts a method or use 2500 and can include 2510 delivering a second revascularization device to a blood vessel of a respective human patient of a first plurality of human patients for retrieving a thrombus; 2525 restoring perfusion to the blood vessel by passing the second revascularization device by, through, or about the thrombus and removing the second revascularization device; and 2530 reducing in-hospital mortality, by the first revascularization device, from approximately a 11.9% to approximately 3.1% for the first plurality of human patients with one or more cerebral occlusions within a predetermined time period. Method or use 2500 can end after step 2530.

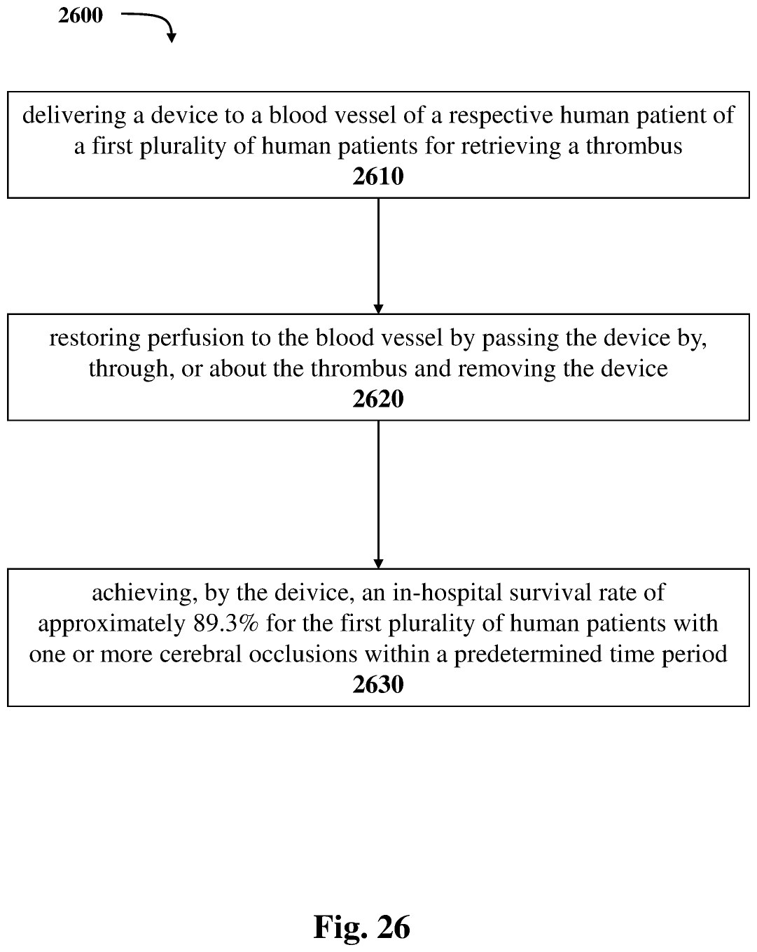

FIG. 26 depicts a method or use 2600 and can include 2610 delivering device to a blood vessel of a respective human patient of a first plurality of human patients for retrieving a thrombus; 2620 restoring perfusion to the blood vessel by passing the device by, through, or about the thrombus and removing the device; and 2630 achieving, by the device, an in-hospital survival rate of approximately a 89.3% for the first plurality of human patients with one or more cerebral occlusions within a predetermined time period. Method or use 2600 can end after step 2630.

The device 200 and related methods of use of this disclosure demonstrated high rates of substantial reperfusion and functional independence in patients with acute ischemic stroke secondary to large-vessel occlusions. The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:

Clause 1: A method or use comprising: delivering a first revascularization device to a blood vessel of a respective human patient of a first plurality of human patients for retrieving a thrombus; restoring perfusion to the blood vessel by passing the first revascularization device by, through, or about the thrombus and removing the first revascularization device; and achieving, by the first revascularization device, approximately a 9.7% in-hospital mortality rate for the first plurality of human patients with one or more cerebral occlusions within a predetermined time period.

Clause 2: The method or use according to Clause 1, further comprising: delivering a second revascularization device to a blood vessel of a respective human patient of a second plurality of human patients for retrieving a thrombus; restoring perfusion to the blood vessel by passing the second revascularization device by, through, or about the thrombus and removing the second revascularization device; and reducing in-hospital mortality, by the first revascularization device, by approximately 11.9% for the first plurality of human patients with one or more cerebral occlusions within a predetermined time period.

Clause 3: The method or use according to Clause 2, comprising: reducing in-hospital mortality, by the first revascularization device, by approximately 3.1% for the first plurality of human patients with one or more cerebral occlusions within a predetermined time period.

Clause 4: The method or use according to Clause 1, wherein the first revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 5: The method or use comprising: delivering a revascularization device to a blood vessel a respective human patient of a plurality of human patients for retrieving a thrombus; restoring perfusion to the blood vessel by passing the revascularization device by, through, or about the thrombus and removing the revascularization device; and achieving, by the revascularization device, approximately 16.1% 30-day all-cause readmission rate for the plurality of human patients with one or more cerebral occlusions within a predetermined time period.

Clause 6: The method or use according to Clause 5, wherein the revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 7: The method or use comprising: delivering a revascularization device to a blood vessel of a respective human patient of a plurality of human patients for retrieving a thrombus; restoring perfusion to the blood vessel by passing the revascularization device by, through, or about the thrombus and removing the revascularization device; and achieving, by the revascularization device, approximately 10.8% 30-day cardiovascular related readmission rate for the plurality of human patients with one or more cerebral occlusions within a predetermined time period.

Clause 8: The method or use according to Clause 7, wherein the revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 9: The method or use comprising: delivering a revascularization device to a blood vessel of a respective human patient of a plurality of human patients for retrieving a thrombus; restoring perfusion to the blood vessel by passing the revascularization device by, through, or about the thrombus and removing the revascularization device; and achieving, by the revascularization device, approximately 4.3% 30-day acute ischemic stroke related readmission rate for the plurality of human patients with one or more cerebral occlusions within a predetermined time period.

Clause 10: The method or use according to Clause 9, wherein the revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 11: The method or use comprising: delivering a first revascularization device to a blood vessel of a respective human patient of a first plurality of human patients for retrieving a thrombus; restoring perfusion to the blood vessel by passing the first revascularization device by, through, or about the thrombus and removing the first revascularization device; and achieving, by the first revascularization device, approximately $45,782 USD total cost of hospitalization care for the first plurality of human patients with one or more cerebral occlusions within a predetermined time period.

Clause 12: The method or use according to Clause 11, further comprising: delivering a second revascularization device to a blood vessel of a respective human patient of a second plurality of human patients for retrieving a thrombus; restoring perfusion to the blood vessel by passing the second revascularization device by, through, or about the thrombus and removing the second revascularization device; and reducing in-hospital mortality, by the first revascularization device, from approximately 11.9% to approximately 3.1% for the first plurality of human patients with one or more cerebral occlusions within a predetermined time period.

Clause 13: The method or use according to Clause 12, comprising achieving, by the first revascularization device, approximately a 9.7% in-hospital mortality rate for the first plurality of human patients with one or more cerebral occlusions within a predetermined time period.

Clause 14: The method or use according to Clause 11, further comprising achieving, by the first revascularization device, approximately 16.1% 30-day all-cause readmission rate for the first plurality of human patients with one or more cerebral occlusions within a predetermined time period.

Clause 15: The method or use according to Clause 11, further comprising achieving, by the first revascularization device, approximately 10.8% 30-day cardiovascular related readmission rate for the first plurality of human patients with one or more cerebral occlusions within a predetermined time period.

Clause 16: The method or use according to Clause 11, further comprising: achieving, by the first revascularization device, approximately 4.3% 30-day acute ischemic stroke related readmission rate for the first plurality of human patients with one or more cerebral occlusions within a predetermined time period.

Clause 17: The method or use according to Clause 11, wherein the first revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 18: A method or use comprising: delivering a device to a blood vessel of a respective human patient of a first plurality of human patients for retrieving a thrombus, the device comprising: a framework of struts forming a porous inner body flow channel and having a tubular main body portion and a distal end, and a framework of struts forming an outer tubular body radially surrounding the tubular main body portion of the inner body during both the collapsed delivery configuration and the expanded deployed configuration; restoring perfusion to the blood vessel by passing the device by, through, or about the thrombus and removing the device; and achieving, by the device, an in-hospital survival rate of approximately 90.3% for the first plurality of human patients with one or more cerebral occlusions within a predetermined time period.

Clause 19: The method or use according to any preceding Clause, the plurality of human patients comprising inclusion criteria defined as patients who underwent a mechanical thrombectomy procedure for acute ischemic stroke between Jul. 1, 2018-Dec. 31, 2019 were identified from the Premier Healthcare Database.

Clause 20: The method or use according to any preceding Clause, the plurality of human patients comprising inclusion criteria defined as patients ≥18 years old at the time of index hospital admission.

Clause 21: A method or use comprising: delivering a first revascularization device to a blood vessel of a respective human patient of a first plurality of human patients for retrieving a thrombus; restoring perfusion to the blood vessel by passing the first revascularization device by, through, or about the thrombus and removing the first revascularization device; and achieving, by the first revascularization device, approximately a 10.7% in-hospital mortality rate for the first plurality of human patients with one or more cerebral occlusions within a predetermined time period.

Clause 22: The method or use according to Clause 21, further comprising: delivering a second revascularization device to a blood vessel of a respective human patient of a second plurality of human patients for retrieving a thrombus; restoring perfusion to the blood vessel by passing the second revascularization device by, through, or about the thrombus and removing the second revascularization device; and reducing in-hospital mortality, by the first revascularization device, by approximately 10.9% for the first plurality of human patients with one or more cerebral occlusions within a predetermined time period.

Clause 23: The method or use according to Clause 22, comprising: reducing in-hospital mortality, by the first revascularization device, by approximately 2.1% for the first plurality of human patients with one or more cerebral occlusions within a predetermined time period.

Clause 24: The method or use according to Clause 21, wherein the first revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 25: The method or use comprising: delivering a revascularization device to a blood vessel a respective human patient of a plurality of human patients for retrieving a thrombus; restoring perfusion to the blood vessel by passing the revascularization device by, through, or about the thrombus and removing the revascularization device; and achieving, by the revascularization device, approximately 9.6% 30-day all-cause readmission rate for the plurality of human patients with one or more cerebral occlusions within a predetermined time period.

Clause 26: The method or use according to Clause 25, wherein the revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 27: The method or use comprising: delivering a revascularization device to a blood vessel of a respective human patient of a plurality of human patients for retrieving a thrombus; restoring perfusion to the blood vessel by passing the revascularization device by, through, or about the thrombus and removing the revascularization device; and achieving, by the revascularization device, approximately 5.9% 30-day cardiovascular related readmission rate for the plurality of human patients with one or more cerebral occlusions within a predetermined time period.

Clause 28: The method or use according to Clause 27, wherein the revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 29: The method or use comprising: delivering a revascularization device to a blood vessel of a respective human patient of a plurality of human patients for retrieving a thrombus; restoring perfusion to the blood vessel by passing the revascularization device by, through, or about the thrombus and removing the revascularization device; and achieving, by the revascularization device, approximately 2.6% 30-day acute ischemic stroke related readmission rate for the plurality of human patients with one or more cerebral occlusions within a predetermined time period.

Clause 30: The method or use according to Clause 29, wherein the revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 31: The method or use comprising: delivering a first revascularization device to a blood vessel of a respective human patient of a first plurality of human patients for retrieving a thrombus; restoring perfusion to the blood vessel by passing the first revascularization device by, through, or about the thrombus and removing the first revascularization device; and achieving, by the first revascularization device, approximately $47,367 USD total cost of hospitalization care for the first plurality of human patients with one or more cerebral occlusions within a predetermined time period.

Clause 32: The method or use according to Clause 31, further comprising: delivering a second revascularization device to a blood vessel of a respective human patient of a second plurality of human patients for retrieving a thrombus; restoring perfusion to the blood vessel by passing the second revascularization device by, through, or about the thrombus and removing the second revascularization device; and reducing in-hospital mortality, by the first revascularization device, from approximately 10.9% to approximately 2.1% for the first plurality of human patients with one or more cerebral occlusions within a predetermined time period.

Clause 33: The method or use according to Clause 32, comprising achieving, by the first revascularization device, approximately a 10.7% in-hospital mortality rate for the first plurality of human patients with one or more cerebral occlusions within a predetermined time period.

Clause 34: The method or use according to Clause 31, further comprising achieving, by the first revascularization device, approximately 9.6% 30-day all-cause readmission rate for the first plurality of human patients with one or more cerebral occlusions within a predetermined time period.

Clause 35: The method or use according to Clause 31, further comprising achieving, by the first revascularization device, approximately 5.9% 30-day cardiovascular related readmission rate for the first plurality of human patients with one or more cerebral occlusions within a predetermined time period.

Clause 36: The method or use according to Clause 31, further comprising: achieving, by the first revascularization device, approximately 2.6% 30-day acute ischemic stroke related readmission rate for the first plurality of human patients with one or more cerebral occlusions within a predetermined time period.

Clause 37: The method or use according to Clause 31, wherein the first revascularization device comprises: a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration.

Clause 38: A method or use comprising: delivering a device to a blood vessel of a respective human patient of a first plurality of human patients for retrieving a thrombus, the device comprising: a framework of struts forming a porous inner body flow channel and having a tubular main body portion and a distal end, and a framework of struts forming an outer tubular body radially surrounding the tubular main body portion of the inner body during both the collapsed delivery configuration and the expanded deployed configuration; restoring perfusion to the blood vessel by passing the device by, through, or about the thrombus and removing the device; and achieving, by the device, an in-hospital survival rate of approximately 89.3% for the first plurality of human patients with one or more cerebral occlusions within a predetermined time period.

Clause 39: The method or use according to any preceding Clause, the plurality of human patients comprising inclusion criteria defined as patients who underwent a mechanical thrombectomy procedure for acute ischemic stroke between Jul. 1, 2018-Dec. 31, 2020 were identified from the Premier Healthcare Database.

Clause 40: The method or use according to any preceding Clause, the plurality of human patients comprising inclusion criteria defined as patients ≥18 years old at the time of index hospital admission.

What is claimed is:

1. A method for use comprising:
delivering a first revascularization device to a blood vessel of a respective human patient of a first plurality of human patients for retrieving a thrombus;
restoring perfusion to the blood vessel by passing the first revascularization device by, through, or about the thrombus and removing the first revascularization device; and
achieving, by the first revascularization device, approximately a 9.7% in-hospital mortality rate for the first plurality of human patients with one or more cerebral occlusions within a predetermined time period.

2. The method according to claim 1, further comprising:
delivering a second revascularization device to a blood vessel of a respective human patient of a second plurality of human patients for retrieving a thrombus;
restoring perfusion to the blood vessel by passing the second revascularization device by, through, or about the thrombus and removing the second revascularization device; and
reducing in-hospital mortality, by the first revascularization device, by approximately 11.9% for the first plurality of human patients with one or more cerebral occlusions compared to the second plurality of human patients within a predetermined time period.

3. The method according to claim 2, comprising:
reducing in-hospital mortality, by the first revascularization device, by approximately 3.1% for the first plurality of human patients with one or more cerebral occlusions compared to the second plurality of human patients within a predetermined time period.

4. The method according to claim 1, wherein the first revascularization device comprises:
a framework of struts forming a porous inner body flow channel and having a tubular main body portion and a distal end; and
a framework of struts forming an outer tubular body radially surrounding the tubular main body portion of the inner body during both a collapsed delivery configuration and an expanded deployed configuration.

5. The method according to claim 1, comprising:
achieving, by the first revascularization device, an in-hospital survival rate of approximately 90.3% for the first plurality of human patients with one or more cerebral occlusions within a predetermined time period.

6. The method according to claim 1, further comprising:
achieving, by the first revascularization device, approximately 16.1% 30-day all-cause readmission rate for the first plurality of human patients with one or more cerebral occlusions within a predetermined time period.

7. The method according to claim 1, further comprising:
achieving, by the first revascularization device, approximately 10.8% 30-day cardiovascular related readmission rate for the first plurality of human patients with one or more cerebral occlusions within a predetermined time period.

8. The method according to claim 1, further comprising:
achieving, by the first revascularization device, approximately 4.3% 30-day acute ischemic stroke related readmission rate for the first plurality of human patients with one or more cerebral occlusions within a predetermined time period.

9. The method according to claim 1, further comprising:
achieving, by the first revascularization device, approximately $45,782 USD total cost of hospitalization care for the first plurality of human patients with one or more cerebral occlusions within a predetermined time period.

10. The method according to claim 9, further comprising:
delivering a second revascularization device to a blood vessel of a respective human patient of a second plurality of human patients for retrieving a thrombus;
restoring perfusion to the blood vessel by passing the second revascularization device by, through, or about the thrombus and removing the second revascularization device; and
reducing in-hospital mortality, by the first revascularization device, by approximately 11.9% to approximately 3.1% for the first plurality of human patients with one or more cerebral occlusions within a predetermined time period.

11. The method according to claim 1, wherein the first plurality of human patients comprise inclusion criteria defined as patients who underwent a mechanical thrombectomy procedure for acute ischemic stroke between Jul. 1, 2018-Dec. 31, 2019 identified from the Premier Healthcare Database.

12. A method for use of reducing in-hospital mortality rate, the method comprising:
delivering a first revascularization device to a blood vessel of a respective human patient of a first plurality of human patients for retrieving a thrombus;
restoring perfusion to the blood vessel by passing the first revascularization device by, through, or about the thrombus and removing the first revascularization device; and
achieving, by the first revascularization device, approximately a 10.7% in-hospital mortality rate for the first plurality of human patients with one or more cerebral occlusions within a predetermined time period.

13. The method according to claim 12, further comprising:
delivering a second revascularization device to a blood vessel of a respective human patient of a second plurality of human patients for retrieving a thrombus;
restoring perfusion to the blood vessel by passing the second revascularization device by, through, or about the thrombus and removing the second revascularization device; and
reducing in-hospital mortality, by the first revascularization device, by approximately 10.9% for the first plurality of human patients with one or more cerebral occlusions compared to the second plurality of human patients within a predetermined time period.

14. The method according to claim 13, comprising:
reducing in-hospital mortality, by the first revascularization device, by approximately 2.1% for the first plurality of human patients with one or more cerebral occlusions compared to the second plurality of human patients within a predetermined time period.

15. The method according to claim 12, wherein the first revascularization device comprises:
a framework of struts forming a porous inner body flow channel and having a tubular main body portion and a distal end; and
a framework of struts forming an outer tubular body radially surrounding the tubular main body portion of the inner body during both a collapsed delivery configuration and an expanded deployed configuration.

16. The method according to claim 12, comprising:
achieving, by the first revascularization device, an in-hospital survival rate of approximately 89.3% for the first plurality of human patients with one or more cerebral occlusions within a predetermined time period.

17. The method according to claim 12, further comprising:
achieving, by the first revascularization device, approximately 9.6% 30-day all-cause readmission rate for the first plurality of human patients with one or more cerebral occlusions within a predetermined time period.

18. The method according to claim 12, further comprising:
achieving, by the first revascularization device, approximately 5.9% 30-day cardiovascular related readmission rate for the first plurality of human patients with one or more cerebral occlusions within a predetermined time period.

19. The method according to claim 12, further comprising:
achieving, by the first revascularization device, approximately 2.6% 30-day acute ischemic stroke related readmission rate for the first plurality of human patients with one or more cerebral occlusions within a predetermined time period.

20. The method according to claim 12, further comprising:
achieving, by the first revascularization device, approximately $47,367 USD total cost of hospitalization care for the first plurality of human patients with one or more cerebral occlusions within a predetermined time period.
